(19) United States
(12) Patent Application Publication
(10) Pub. No.: US 2022/0378710 A1
(43) Pub. Date: Dec. 1, 2022

(54) Therapeutic Gas Microfoam for Skin Recovery

(71) Applicant: Truly Oxygen, Inc., Boulder, CO (US)

(72) Inventors: Paul MOUNTFORD, Boulder, CO (US); Paul MARCHANDO, Highlands Ranch, CO (US); Jeffrey GEORGE, Boulder, CO (US); Lyndsi ANSAY, Centennial, CO (US); Ian VELASCO, Anchorage, AK (US); Robert T. SCRIBNER, Boulder, CO (US); Gabrielle MELLI, Fort Collins, CO (US)

(21) Appl. No.: 17/882,334

(22) Filed: Aug. 5, 2022

Related U.S. Application Data
(63) Continuation-in-part of application No. 17/077,897, filed on Oct. 22, 2020.
Application No. 17/077,897 is a continuation of application No. PCT/US19/28526, filed on Apr. 22, 2019.
(60) Provisional application No. 63/229,966, filed on Aug. 5, 2021.
(60) Provisional application No. 62/661,336, filed on Apr. 23, 2018.

(51) Int. Cl. A61K 9/50; A61K 33/00

(52) U.S. Cl. CPC A61K 9/5015; A61K 33/00; A61K 9/0014

(57) ABSTRACT

Disclosed are improved devices, systems, methods and compositions containing therapeutic lipid monolayer stabilized gas microfoam(s) that can be applied to the skin after a treatment which “damages” or otherwise modifies one or more skin layers, which can improve a patient's recovery time, reduce swelling, scarification and/or callous formation, reduce skin sensitivity and/or lead to a more desirable appearance in a shorter period of time than existing skin treatments.

### CROSS-REFERENCE TO RELATED APPLICATIONS

This application claims the benefit of U.S. Provisional Patent Application Ser. No. 63/229,966 entitled “Therapeutic Gas Microfoam for Skin Recovery,” filed Aug. 5, 2021. This application is also a continuation-in-part application of U.S. patent application Ser. No. 17/077,897 entitled “Oxygenated Skin Lotion,” filed Oct. 22, 2020, which is a continuation application of PCT Patent Application Serial No. PCT/US19/28526 entitled “Oxygenated Skin Lotion,” filed Apr. 22, 2019, which claims the benefit of U.S. Provisional Patent Application Ser. No. 62/661,336 entitled “Oxygenated Skin Lotion,” filed Apr. 23, 2018. The disclosures of all of these documents are each incorporated by reference herein in their entireties.

### TECHNICAL FIELD

The invention relates to improved devices, systems and methods of delivering oxygen and/or other therapeutic substances into the skin of a living organism, such as a mammal and/or human patient. More specifically, disclosed are a variety of ointments, creams, lotions, waters, extracts, pastes, powders, gels, tinctures, dressings and/or other compounds that utilize microbubble carriers to desirably enable and/or facilitate the transport of oxygen and/or other therapeutic substances into, through and/or around the stratum corneum (i.e., the horny layer) of the skin and epidermis via a variety of penetration processes in order to increase the oxygen concentration in the dermal area and/or adjoining tissues to desirably activate various metabolic processes. In one exemplary embodiment, therapeutic gas microfoams can be highly useful as topical creams and ointments for skin recovery after non-invasive, chemical, abrasive, radiofrequency, laser, and invasive skin care cosmetic and therapeutic procedures.

### BACKGROUND OF THE INVENTION

Oxygen is one of the basic essentials for sustaining life and comprises approximately 20.95% of dry atmospheric air. While humans and other mammals are capable of passively absorbing some levels of oxygen directly from the atmosphere (via upper layer skin cells and the cells in the front surface of the eyes, for example), human and/or mammalian bodies have a huge demand for oxygen, and thus their need for lungs which actively pull in oxygen and transfer it to the blood, allowing the body to transport oxygen to various cells throughout the body.

The skin is the only major organ besides the lungs that is directly exposed to atmospheric oxygen. Because skin is exposed directly to the air, the outer layers of the skin can absorb oxygen directly from the atmosphere. According to at least one study, the upper skin layers to a depth of 0.25-0.40 mm are almost exclusively supplied by externally absorbed oxygen, whereas the oxygen transport of the blood has a minor influence on these layers. In many cases, the amount of this externally supplied oxygen that makes it into the anatomical layers below the skin is negligible, so that most of the cells in a human or mammalian body get their oxygen directly from the blood.

Apart from the stratum corneum, oxygen is consumed in all layers of the epidermis and dermis. The oxygen demand of these layers can be partially satisfied by the blood: the dermis exhibits a vasculature that is arranged in two tiers that are parallel to the skin surface. The superficial plexus between the papillary and the upper reticular dermis deep plexus in the lower reticular dermis are connected by perpendicularly orientated communicating vessels. Arcades of capillaries loop upwards into the papillae from the subpapillary plexus. In contrast, the epidermis has no vasculature, but is exposed directly to the atmosphere.

Although most research into the changes in skin with age focus on the unwelcome aesthetic aspects of the aging skin, skin deterioration with age is more than a merely cosmetic problem. The skin ages in both men and women through parallel internal and external processes, which contribute simultaneously to a progressive loss of skin integrity. Aged skin undergoes progressive structural and functional degeneration that leaves it prone to a wide variety of bothersome, debilitating, and possibly even fatal conditions and diseases, including eczema, asteatotic eczema, contact and allergic dermatitis, seborrheic dermatitis, autoimmune diseases with cutaneous manifestations, seborrheic keratoses, and various forms of neoplasms, such as basal and squamous cell carcinoma and malignant melanoma. Although mortality from skin disease is primarily restricted to melanoma, dermatological disorders are ubiquitous in older people with a significant impact on quality of life. The structural and functional deterioration of the skin that occurs with age has numerous clinical presentations, ranging from benign but potentially excruciating disorders like pruritus to the more threatening carcinomas and melanomas. In addition, cosmetic changes in the aging skin can involve a variety of conditions, including the overall facial skin appearance, as well as skin brightness, evenness, firmness, pore size, radiance, fine lines, coarse wrinkles, and blotchiness or dyspigmentation.

A loss of both function and structural stability in skin proceeds unavoidably as individuals age, which is the result of both intrinsic and extrinsic processes, which contribute simultaneously to a progressive loss of skin integrity. Intrinsic aging proceeds at a genetically determined pace, primarily caused by the buildup of damaging products of cellular metabolism as well as an increasing biological aging of the cells. Physiological changes in aged skin include structural and biochemical changes as well as changes in neurosensory perception, permeability, response to injury, repair capacity, and increased incidence of some skin diseases. Although the number of cell layers remains stable, the skin thins progressively over adult life at an accelerating rate. The epidermis decreases in thickness, particularly in women and particularly on the face, neck, upper part of the chest, and the extensor surface of the hands and forearms. Thickness decreases about 6.4% per decade on average, with an associated reduction in epidermal cell numbers. Keratinocytes, as skin ages, change shape, becoming shorter and fatter, while corneocytes become bigger as a result of decreased epidermal turnover. Enzymatically active melanocytes decrease at a rate of 8% to 20% per decade, resulting in uneven pigmentation in elderly skin. Although the number of sweat glands does not change, sebum production decreases as much as 60%.

The most consistent structural change in aged skin is a flattening of the dermo-epidermal junction by more than a third, which occurs as a result of the loss of dermal papillae as well as a reduced interdigitation between layers. This flattening, observable by scanning electron microscopy beginning in the sixth decade, results in less resistance to shearing forces and an increased vulnerability to insult. The smaller contiguous surface between the two layers also creates a reduced cellular supply of nutrients and oxygen, and an increased risk of dermo-epidermal separation, a process which may be the mechanism by which wrinkles form. A reduction of the natural water and fat emulsion on the skin is also observed, as is water content in the stratum corneum. Global lipid content of the aged skin is reduced as much as 65%. Changes in the amino acid composition in aged skin may reduce the amount of cutaneous natural moisturizing factor, thereby decreasing its capacity for water binding, and profound changes in barrier integrity can occur, despite indications that barrier function in aged skin under normal conditions often appears normal.

Wrinkles are caused by a variety of environmental factors including smoking, dietary intake, and UV exposure, as well as the body's intrinsic aging process. Intrinsic aging of the skin causes decreased production of fibroblasts, collagen, and elastin leading to wrinkles and loss of skin elasticity. The most prominent cause of skin aging is UV exposure, which has been shown to be responsible for 80% of visible signs of skin aging such as irregular pigmentation and wrinkles. Wrinkles caused by intrinsic aging present as “fine” lines, while wrinkles caused by photoaging are characterized as “coarse”, likely due to the thickening of skin associated with UV exposure.

In many cases, individuals seeking to reduce the effects of aging on the skin may undergo non-invasive chemical or abrasive skin procedures or even invasive skincare procedures, performed by trained skincare professionals, such as (but not limited to) estheticians, aestheticians, dermatologists, dentists, facial plastic surgeons and the like. After such skin treatments, the patient's skin is often irritated, inflamed and/or appears red in color, and natural healing of the skin commonly requires 3 days to 2 weeks or longer to occur before full recovery. During this recovery period, the skin can be unusually sensitive to abrasion, irritation and/or environmental effects (i.e., natural UV light and sunburns) and must often be covered or protected to prevent additional damage or scarring of the treated tissues.

### BRIEF SUMMARY OF THE INVENTION

The present invention includes the realization of a need for oxygen delivery systems, devices, techniques and/or methods that may partially and/or fully supply oxygen and/or other nutrients directly to and/or through contacted tissues, such as skin surfaces, in a non-invasive, easily portable, safe and easily-used manner. In at least one exemplary embodiment, a skin preparation comprising an oxygen microbubble foam layer can be applied to an epidermal surface after an abrasive or chemical “peel” treatment to protect the damaged skin surface and/or facilitate lipid and/or oxygen delivery directly to and/or through the epidermis and deeper down into the dermal tissues, thereby dramatically improving recovery time and reducing visible redness. Desirably, the oxygen microbubble foam layer will provide needed oxygen and/or nutrients and may optionally be further capable of absorbing and/or otherwise disposing of waste products from skin metabolism and repair, such as carbon dioxide and/or urea.

In various exemplary embodiments, a method of providing oxygenation to an individual's skin surfaces can include the topical application of a compound including microbubbles containing oxygen and/or other substances (including oxygen microbubbles or OMBs) to portions and/or sections of the epidermis (i.e., the skin) of the individual—in many cases primarily to exposed skin such as the face, hands, torso, arms and legs of the individual in the form of a cream, gel, lotion and/or cosmeceutical formulation. The oxygen microbubble (OMB) carrier may comprise oxygen gas filled bubbles having a shell composed of an amphiphilic surfactant phospholipid monolayer or cross-linked polymers or a combination of phospholipids and polymers, and may include other substances to enable and/or facilitate transfer of gases and/or other compounds into and/or out of the microbubbles. Through the presence of the oxygen microbubbles that are in contact with and/or in proximity to the skin surface, oxygen and/or carbon dioxide exchange (and flow of other nutrients and/or wastes) may occur. An overall improvement in the health and quality of the epidermis and/or other skin layers may be achieved through use of the invented system and methods.

In various embodiments, the OMB formulation may include compounds and/or other features which “target” and/or otherwise demonstrate a preference for one or more skin types and/or regions of the epidermis for delivery of one or more OMB payloads, including oxygen. For example, “oily skin” may allow for significantly more oxygen flow into and/or through the skin from the OMB compound than a comparable region of “dry skin, even of a single individual. In such a case, a first formulation (for application to the oily skin region) may contain a lower amount of hydrating or other compounds than a second formulation (for application to the dry skin region).

In various embodiments, the OMBs may deliver oxygen to one or more specific locations of the epidermis, or the delivery of oxygen and/or other compounds may occur at multiple locations and/or along the entirety of the applied surface of the skin and/or various portions thereof. Where an individual OMB has delivered some portion of its oxygen payload (and/or other compounds) to the individual's anatomy, the individual OMB may “destruct” (i.e., breakdown of the microbubble shell typically in response to shear forces), the OMB may reduce in size to become a smaller microbubble, and/or the OMB may increase in size via absorption and/or incorporation of other substances (i.e., carbon dioxide, other gases and/or metabolic wastes). The OMB may also “destruct” or otherwise alter in size and/or shape through the absorption of the lipid shell, causing the OMB to break down and expose the oxygen or other contents to the skin.

In various embodiments, the amphiphilic phospholipid monolayer shell variation of an exemplary OMB embodiment can have similar composition to lung surfactant and may require comparable physical properties, such as rapid adsorption to and mechanical stabilization of the gas/liquid interface and high gas permeability. Thus, OMBs can be designed to mimic the mechanical and gas transport properties of the alveolus to deliver the oxygen payload. By transport into and/or through the skin, phospholipid monolayer, cross-linked polymer or mixed phospholipid-polymeric stabilized OMBs will desirably provide oxygen for uptake through tissues to the underlying skin layers and/or even to the bloodstream. In addition, any “unused” and/or waste filled microbubbles can easily be removed from and/or naturally flake off the surface of the skin in the natural progression, as well as any component materials from OMBs that “burst” or otherwise destruct or are released during such activities.

### BRIEF DESCRIPTION OF THE SEVERAL VIEWS OF THE DRAWINGS

FIG. 1 depicts one embodiment of an exemplary Oxygen Micro Bubble (OMB);

FIG. 2 graphically depicts one exemplary embodiment of an oxygen microbubble production distribution;

FIG. 3 graphically depicts exemplary microbubble oxygen content over time;

FIG. 4 graphically depicts an exemplary reduction of wrinkle depth by application of a topical oxygen-rich cream;

FIG. 5 depicts one exemplary embodiment of an OMB formulation that can facilitate transfer of oxygen into skin layers to assist with regulation of angiogenesis activation;

FIG. 6 depicts a simplified view of an exemplary skin anatomy;

FIG. 7 depicts exemplary pathways for molecules to penetrate the stratum corneum (SC) of the skin;

FIG. 8 depicts an exemplary penetration pathway for oxygen from a microbubble formulation containing Oxygen when applied to a skin surface;

FIG. 9 graphically depicts penetration depth versus time for various OMB formulations applied to a skin surface;

FIG. 10A depicts one exemplary ingredient for an OMB formulation for application to a skin surface;

FIG. 10B depict various ratios of ingredients in exemplary control and test formulations for topical skin application;

FIG. 11 depicts a flowchart of various exemplary production steps for producing a topical OMB formulation;

FIG. 12 depicts an exemplary method for producing a topical OMB cream;

FIG. 13 depicts a series of five experimental OMB cream samples and five control cream samples;

FIG. 14 depicts various characteristics of the experimental and control samples of FIG. 13;

FIG. 15 graphically depicts penetration depth versus time for the experimental OMB cream formulations of FIG. 13 applied to a skin surface;

FIG. 16 depicts an exemplary microbubble stability test setup;

FIG. 17 depicts emulsion stability for two exemplary OMB formulations;

FIG. 18 depicts a flowchart of exemplary method steps for producing OMBs;

FIG. 19 depicts a flowchart of exemplary method steps for producing an OMB lotion or skin cream;

FIG. 20 depicts diffusion forces driving a targeted gas species from a region of higher gas concentration to a region of lower gas concentration;

FIG. 21 depicts various gas transport modalities spread across an exemplary complex skin surface interface;

FIG. 22 depicts before and after images of one exemplary experimental use of a microfoam cream containing oxygen and lipids;

FIG. 23 depicts before and after images of another exemplary experimental use of a microfoam moisturizing compound containing oxygen and lipids; and

FIG. 24 depicts an exemplary therapeutic payload incorporated into an aqueous phase, a gas phase and/or contained or attached to a lipid monolayer of an exemplary microfoam compound.

### DETAILED DESCRIPTION OF THE INVENTION

The drawings and the following description relate to preferred embodiments by way of illustration only. It should be noted that from the following description, alternative embodiments of the components and methods disclosed herein will be readily recognizable as viable alternatives that may be employed in one skilled in the art.

Oxygen and lipid have both been proven to improve inflammation and wound healing in the skin. Oxygen, a gas at Standard Temperature and Pressure (STP), is most commonly delivered to a wound surface by placing the wound within a pressurized oxygen chamber (hyperbaric oxygen therapy—HBOT). Lipids are most commonly delivered to a tissue surface such as the epidermis via topical application, and to the dermis via topical application post-microchannel formation—for example (but not limited to) microneedling, skinpen, etc.). However, not only does a hyperbaric oxygen treatment require the patient to remain stationary (and often within the chamber) during an extended period of therapy treatment time within a large and inconvenient to use chamber, but gaseous oxygen transport of bulk oxygen through an epidermal layer is highly inefficient, and such transport can easily be blocked by the presence of applied lipids or creams on the skin surface. In contrast to existing treatments, the oxygen microbubble foams and creams presented herein will desirably allow skincare professionals to deliver oxygen and lipid to the epidermis and dermis post skincare treatment without the need for a pressurized chamber to improve patient recovery time, appearance, and outcome.

Currently, patients who seek out skin rejuvenation and anti/reverse-aging procedures must typically allow for extended skin recovery and healing time to achieve the healing and appearance they desire. Aside from the inconvenience and various limitations inherent with the healing process, many individuals have a desire to conceal or hide the appearance of injury from “cosmetic” procedures they receive. For such individuals, the accelerated healing times associated with the present inventions can represent an important and highly valuable improvement over the current state of the art. The disclosed lipid monolayer stabilized gas microfoam compositions applied to the skin after treatment can dramatically reduce a patient's recovery time and lead to a more desirable appearance in a shorter time period, and with less chance of skin thickening or scarification. The disclosed embodiments allow therapeutic delivery of a gas or other compositions to the skin without the need of an enclosed pressurized chamber. Some exemplary procedures for which the disclosed embodiments can provide a benefit can include, but are not limited to, Morpheus8 RF (radiofrequency) facial or body fractional remodeling and Microneedling, Hydrafacial, cleansing facial, permanent makeup and tattoos, waxing, laser treatment, chemical peels, face lifts, neck lifts, rhinoplasty, brow lift, lip augmentation, lip lift, dermapen, helios skin resurfacing, V Beam pulsed dye laser, dermal fillers, Icon skin resurfacing, laser skin resurfacing, etc. The disclosed therapies can desirably improve skin recovery and healing time to make the outcome of these procedures much quicker and more desirable.

In various embodiments, a composition containing therapeutic lipid monolayer stabilized gas microfoam(s) can be applied to the skin after a treatment which “damages” or otherwise modifies one or more skin layers, which can improve a patient's recovery time, reduce swelling, scarification and/or callous formation, reduce skin sensitivity and/or lead to a more desirable appearance in less time.

In various exemplary embodiments, a method of providing supplemental oxygenation to various skin layers of an individual can include the topical application of microbubbles containing oxygen and/or other substances (including oxygen microbubbles or OMBs) to surfaces of the skin of the individual. The oxygen microbubble (OMB) carrier may comprise oxygen filled bubbles having a shell composed of an amphiphilic surfactant phospholipid monolayer, a cross-linked polymer, or a combination of phospholipids and polymers, in combination with other compounds to form an ointment, cream, lotion, water, extract, paste, powder, gel, tincture, dressing, a cosmeceutical formulation (i.e., a cosmetic product with medicinal or drug-like benefits from a special ingredient or additive) and/or other topically applied amalgam. In at least one variation, the amphiphilic phospholipid monolayer shelled OMB can have a similar composition to lung surfactant and requires comparable physical properties, such as rapid adsorption to and mechanical stabilization of the gas/liquid interface and high gas permeability. Thus, OMBs can be also designed to mimic the mechanical and gas transport properties of the alveolus to deliver an oxygen payload. By transport into and/or through the skin wall (specifically into, through and/or around the stratum corneum), the phospholipid monolayer OMBs will desirably provide oxygen for uptake via cells in the surface skin layers, as well as potentially by the underlying bloodstream. Similarly, biocompatible polymer shelled microbubbles can readily be delivered to the skin surface via topical application and are able to deliver oxygen and ultimately easily removed from the anatomy, if necessary. In various embodiments, the gas-filled microbubble suspensions described herein can be formulated in a manner suitable for topical administration, e.g., as a liquid and semi-liquid preparation that can be absorbed by the skin. Examples of a liquid and semi-liquid preparation include, but are not limited to, topical solutions, liniments, lotions, creams, ointments, pastes, gels, and emugels. The preparation may be applied with an exterior lining which is not gas permeable in order to promote transfer of the contents to the skin and not to the atmosphere.

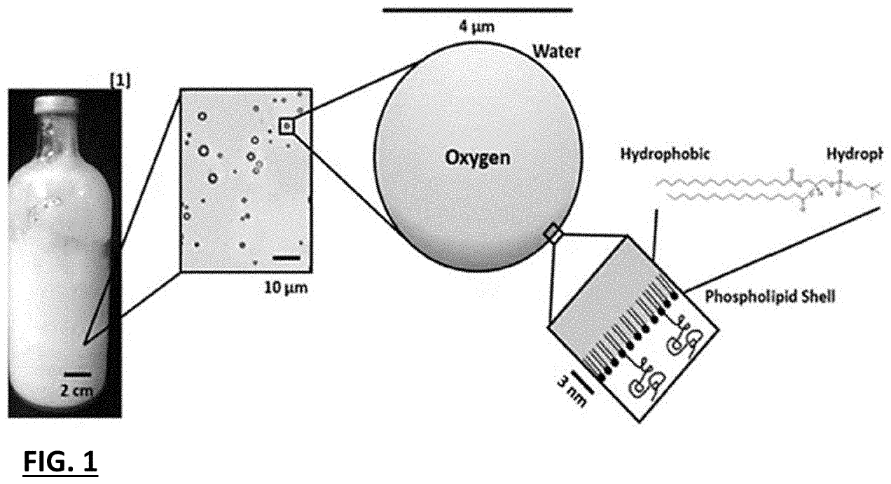

FIG. 1 depicts one embodiment of an exemplary Oxygen Micro Bubble (OMB). In this embodiment, the OMB comprises an oxygen gas encapsulated by a phospholipid shell, with an average diameter on the order of 1 to 10 microns (with an approximately 4-micron diameter bubble depicted).

In an adult human, the total surface area of the skin has been estimated from 2 to 3 square meters (in some studies). In various embodiments, OMB's may be applied topically to the skin surface of an individual's body where the OMBs may contact the tissues and may transfer a gas, compound and/or other payload into and/or through the cells of the skin for local treatment and/or systemic treatment and/or potential distribution via the blood stream and/or lymphatic system.

In various embodiment, the OMB formulation may also provide pain relieving effects. For example, phospholipid monolayer microbubbles may be used in combination with other gases and additives to provide an optimum composition for specific physiologic effects. Anesthetic gases delivered by topical diffusion and/or absorption from the phospholipid monolayer microbubbles may (1) provide enhanced local anesthetic saturation levels for mammals; (2) provide enhanced anesthetic performance by delivery of anesthetic agents to the body. In various embodiments, a variety of anesthetic compounds may be delivered in conjunction with the OMB formulation, which may include substances to augment anesthetic compounds provided for certain medical purposes as well as agents that may enable and/or enhance anesthetic effects for pain relief, surgical interventions, dental treatments, and relief of physical discomfort.

According to the invention, OMBs can be designed for high oxygen carrying capacity, high oxygen delivery rate and sufficient stability for storage and transport. Direct oxygenation by applying OMBs to the surface of the skin or other tissues is a radical change from existing oxygen delivery platforms.

As used herein, microbubbles generally refer to micron-sized (e.g., in the range of 1 um to 1000 um in diameter) substantially spherical gas-filled particles in solution that are stabilized by an organic coating at the gas-liquid interface. The stability, gas diffusion properties, and biocompatibility of microbubbles can be controlled via the formulation of the coating material (i.e., the microbubble shell). Customizing the stabilizing shell of the microbubbles can allow fabricated microbubbles to be stored for later use. Alternatively, the microbubbles may be used immediately after fabrication. In such cases, the coating material may be sufficiently stable as to allow the microbubble to deliver its gas payload to an intended target (e.g., into and/or through the skin layers of a patient).

According to various features of the present invention, OMBs can be designed and constructed for high oxygen carrying capacity, high oxygen delivery rate and/or sufficient stability for storage and transport. The procedure for delivery of OMBs to the surface of the skin is simple and straightforward, and requires little or no special equipment to accomplish. In addition, larger microbubbles (about 10-25 um diameter) can be utilized in the various formulations herein without fear of adverse effects, because they are separated by the exterior skin layers from the internal tissues and vasculature. Thus, it is contemplated that microbubbles may be between 1-100 um in diameter and even between 1-500 um in diameter. In addition, mixtures of microbubbles may comprise microbubbles of different sizes. The sizes of the OMBs contained within any one mixture may be only smaller microbubbles, only larger microbubbles or a combination of both smaller and larger microbubbles.

In various embodiments, the delivery of a gas contained within the phospholipid and/or polymeric monolayer shell microbubble may include gases other than oxygen, or in combination with oxygen, including nitrogen, hydrogen, fluorine or fluorinated gases, chlorine, helium, neon, argon, krypton, xenon and/or radon in varying compositions according to the desired therapeutic effect. Hyperoxic mixes may be used as a means to draw dissolved inert gases from the body. In other embodiments, the microbubbles may include gaseous compounds other than oxygen, or in combination with oxygen or other elements, including NO2 (nitrous oxide), CO2 (carbon dioxide) CH4 (methane), NH3 (ammonia), HCN (hydrogen cyanide), CO (carbon monoxide), NO (nitric oxide), C2H6 (ethane), PH3 (phosphine), H2S (hydrogen sulfide), HCl (hydrogen chloride), CO2 (carbon dioxide), N20 (dinitrogen oxide), C3H8 (propane), NO2 (nitrogen dioxide), 03 (ozone), C4H10 (butane), SO2 (sulfur dioxide), BF3 (boron trifluoride, Cl2 (chlorine), CF2Cl2 (dichlorodifluoromethane) and/or SF6 (sulfur hexafluoride) in varying compositions according to the desired therapeutic effect.

The ability to deliver oxygen from OMBs via topical application may also have significant clinical implications. For example, where hypoxia of a tissue region occurs (due to vascular obstruction and/or constriction or due to other causes) the application of a topical OMB formulation containing readily accessible oxygen-bearing microbubbles may prevent injury and/or necrosis of surface and/or subsurface tissues for varying lengths of time. Such topical applications could include the delivery of supplemental oxygen in lower concentrations via topical application (i.e., less than 25% of physiologic demand or less than 20% of physiologic demand or less than 15% of physiologic demand or less than 10% of physiologic demand or less than 5% of physiologic demand or less than 4% of physiologic demand or less than 3% of physiologic demand or less than 2% of physiologic demand or less than 1% of physiologic demand).

Phospholipid monolayer or cross-linked polymer or phospholipid-polymeric microbubbles may be used in combination with other fluids and additives to provide an optimum composition for specific physiologic effects. Oxygen delivered by topical application of a microbubble suspension may promote healing of wounds, burns, or other injuries where oxygen is of importance to reduced healing or recovery time and/or provide enhanced delivery of oxygen and/or other compounds (i.e., sucrose, glucose, caffeine, or other agents) to the body. In various embodiments, a variety of compounds may be delivered in conjunction with the OMB formulation, which may include substances to encourage and/or facilitate the passage of oxygen and other gases into and/or out of the skin, as well as substances that may enable and/or enhance absorption of OMB constituents.

In another embodiment, the OMB formulation may be applied topically at the site of a wound in an effective amount to enhance wound healing. The LOM formulation can be applied topically on a continuous basis to infected wounds, as is the case with necrotizing fasciitis, in which the creation of hyperoxic wound conditions is known to decrease mortality and amputation from the disease. The conditions created by the OMB formulation are likely to be unsuitable for bacterial growth, especially so in the case of anaerobic bacterial organisms. This therapy would complement traditional antimicrobial agents (antibiotics) by providing a new mechanism for bacterial killing which would not be amenable to traditional mechanisms for bacterial resistance.

In some exemplary embodiments, administration of the OMB formulation may be associated with one or more additional compounds that modify the individual's skin tissue layers (or portions thereof) to facilitate the durability, passage and/or absorption of, and/or to enable and/or facilitate absorption of OMB constituents by the skin. For example, it may be desirous to alter the humidity levels of the skin surface prior to and/or during application of the OMB formulation, as the normal levels of skin humidity may reduce and/or limit the durability of the microbubbles and/or negatively affect the ability of the OMBs to transfer oxygen into and/or through the skin surface. Such alteration might be accomplished by the application of moisturizing formulation just prior to application of the OMB formulation, or the moisturizer may be incorporated into the OMB formulation for concurrent and/or subsequent application. In various embodiments, one or more components of the microbubble itself (i.e., lipids and/or saline components) might accomplish and/or facilitate “wetting” of the skin surface and/or sub-skin structures in various manners to facilitate trans-cutaneous passage and/or absorption of oxygen and/or other materials.

Trans-cutaneous administration of pharmaceuticals and other therapeutic materials has considerable advantages in terms of patient acceptability, reducing the risk of infection, cost and the quantity of material that can be delivered. Frequently, however, topical administration may be associated with inefficient delivery and/or poor bioavailability, but the use of shell-stabilized oxygen microbubbles for topical application provides a stable delivery medium which delivers oxygen without requiring inhalation, ingestion and/or injection of the oxygen-containing media.

In various embodiment, microbubbles may be employed which utilize surfactant and lecithin-based mixtures (which may provide varying levels of effectiveness in various alternative embodiments). However, using known and isolated amphiphilic phospholipids and biocompatible polymers as the shell material in OMBs desirably provides a mixture composition that is fully understood, thereby allowing for the behavior of the OMBs to be relatively predictable. This enhanced OMB behavior predictability allows the OMBs to be fabricated for greater stability, control of oxygen release, manufacturability, improved storage and handling, and greater efficacy in oxygen delivery. Additionally, OMBs on the order of 1-1000 um in diameter experience a lower internal Laplace pressure (responsible for driving dissolution) than OMBs 1-999 nm in diameter range, allowing the micron-sized OMBs to persist longer on the skin surface.

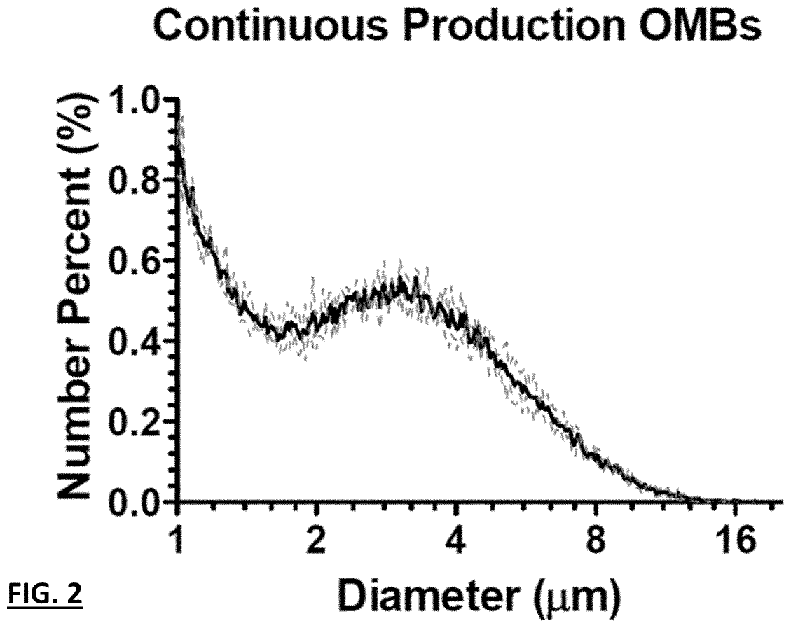

FIG. 2 depicts a graph of one exemplary embodiment of oxygen microbubbles, which can be produced using a variety of production methods and/or techniques, including continuous production and/or batch production. If desired, the OMBs can be produced immediately prior to use, or they can be manufactured and stored for extended periods of time prior to use in the various embodiments described herein. In at least one exemplary embodiment, the size of the OMBs utilized herein can be primarily distributed between 1 and 10 microns (um) in diameter, although larger and/or smaller microbubbles and/or microbubble distributions can be utilized in a variety of the disclosed embodiments with varying results.

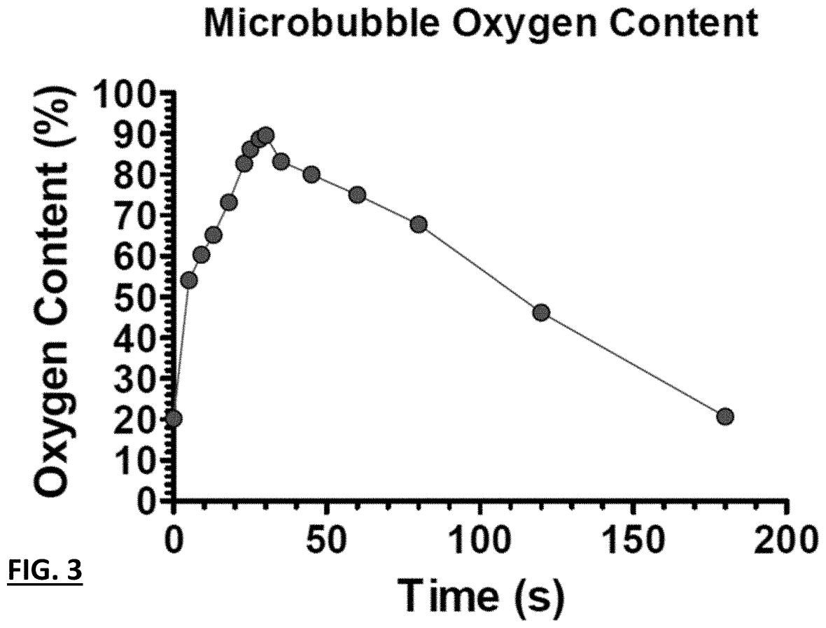

FIG. 3 depicts a graph of microbubble oxygen content over time, specifically an amount of oxygen being released from within phospholipid microbubbles through a diffuse oxygen sensor. For measurement, 10 mL of phospholipid OMBs were broken down in a gas tight syringe via cyclic pressurization. Once full OMB destruction was observed (i.e., no foam, only liquid left in syringe, ^{˜}1-2 mL of liquid volume), the syringe was connected to the diffuse oxygen sensor, allowing the oxygen to pass through the sensor and be measured. The sensor was stored in a natural air environment prior to measurement (^{˜}20% oxygen).

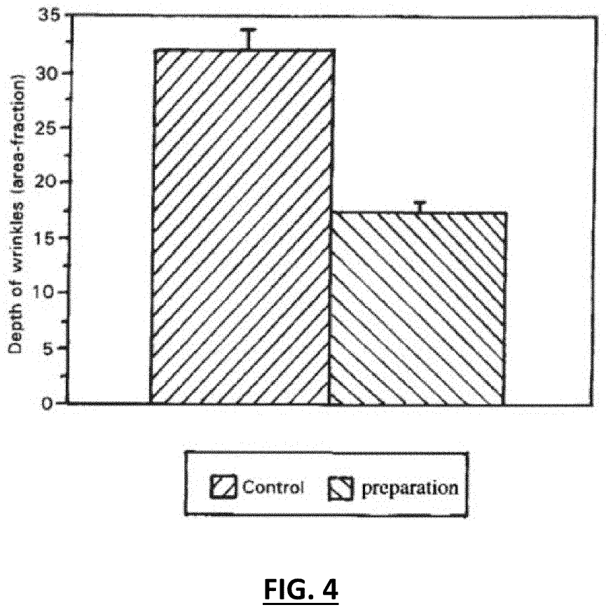

Increasing the local available oxygen within the surface skin levels can dramatically improve the cosmetic appearance of the skin. For example, FIG. 4 depicts an exemplary reduction of wrinkle depth by application of a topical oxygen-rich cream. In this embodiment the cream was applied to the skin twice a day for fourteen (14) days, with the wrinkle depth in the treated area reduced by almost 50% as compared to an untreated control skin area on the same individual. Increased oxygen can also improve the clinical condition of the skin, such as the use of hyperbaric oxygen treatments to decrease the clinical severity of eczema. But such hyperbaric treatments require an expensive hyperbaric chamber, the presence of trained personnel to operate equipment and manage clinical complications (which can include serious injury and/or death in extreme cases) and significant restrictions on the individual's activity during treatment—all of which are obviated by the present invention, as the application of a topical OMB cream to a skin surface can augment, improve, or potentially duplicate the effects of hyperbaric oxygen treatments over the treated area.

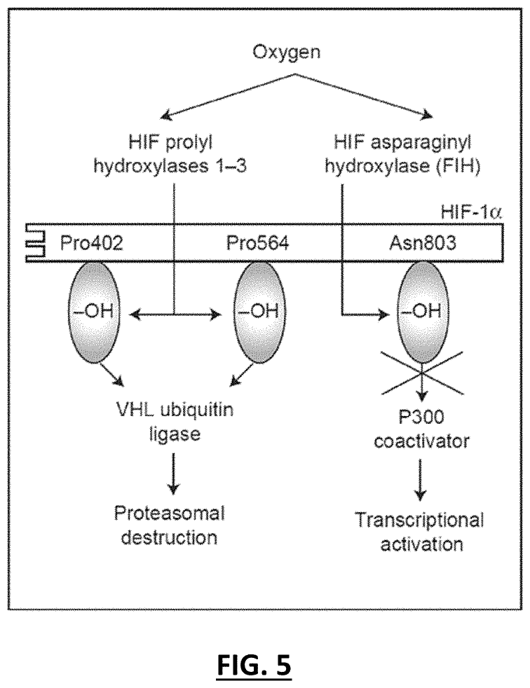

FIG. 5 depicts one exemplary embodiment of an OMB formulation that facilitates the transfer of oxygen into skin layers to assist with regulation of angiogenesis activation. In this embodiment, the effects of angiogenesis are counteracted by hyperoxia, which affects the HIF-1α pathway. Increased rates of angiogenesis are correlated with increased wrinkling in the skin. UV-B radiation causes angiogenesis through several pathways including increasing type 1 collagenase which enzymatically degrades collagen and through matrix metalloproteinases (MMPs) which degrade the basement membranes that anchor layers of the skin together. Neither of these pathways appear to be significantly affected by varying oxygen levels. The third pathway, involving hypoxia inducible factor-1 (HIF-1) is of the most interest because it is counteracted by hyperoxic conditions. HIF-1 is expressed at higher rates under UV-B exposure and when accumulated in cells, HIF-1 recognizes the promoter region of vascular endothelial growth factor (VEGF) and the gene transcription of VEGF is enhanced, causing angiogenesis. However, when oxygen is present, HIF-1 subunits are subject to ubiquitination which tags the protein for proteasomal degradation and prevents VEGF transcription. Thus, increased oxygen levels prevent angiogenesis and skin wrinkling from UV-B.

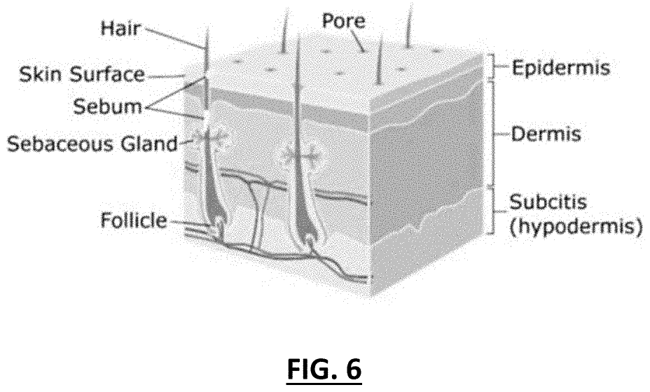

FIG. 6 depicts a simplified view of the skin anatomy, wherein the skin surface includes an exterior epidermis, a dermis and a subcitis or hypodermis. In the skin, the epidermis comprises a basal layer and the stratum corneum, with stratum corneum comprising dead keratinocytes, which are the main barrier or “bottleneck” to the mass transfer of oxygen into the skin. The next layer of the skin, the dermis, includes sebaceous and sweat glands, nerve fibers and living keratinocytes. In addition, the skin possesses a blood supply that runs along the bottom of, and extends partially into, the dermis.

The main function of the skin is to be a barrier between the body and the mechanical, chemical, and microbial influences of the outside world. The stratum corneum (SC) is the outermost and most difficult to penetrate layer of the skin for external materials. It is composed of densely layered dead and dehydrated keratinocytes which are “glued” together in highly ordered lipid layers. The SC is often described as a brick wall with the keratinocytes representing the bricks. There are three known pathways for molecules to penetrate the SC—the intercellular pathway, the transcellular pathway, and the transappendageal pathway.

In the intercellular pathway, molecules pass in between the brick-like keratinocytes. Since the cells are so tightly packed, molecules passing through the SC this way must be 500 kDa or smaller, be sufficiently soluble in oil, and have a high partition coefficient. A 500 kDa molecular weight cut off (MWCO) ultrafilter corresponds to a pore diameter of about 20 nm. OMBs have a number weighted mean diameter of about 3.4±1.9 um [1], much larger than 20 nm, so larger OMBs will typically not pass the stratum corneum intercellularly.

In the transcellular pathway, molecules are absorbed and then secreted by keratinocytes transporting them through the skin, but there is very little evidence supporting this as a viable route for any substance without additional chemical facilitators.

The final skin uptake pathway is the transappendageal pathway in which substances are transported from the epidermis to the dermis through hair follicles, sebaceous glands, and sweat glands. Research has shown that while particles with diameters of 0.75 and 1.5 um may be able to enter hair follicles superficially, they typically do not penetrate deeply enough to diffuse through the follicle epithelium and into the dermis. Since the OMBs are generally larger than 1.5 um, it is not expected that the OMBs themselves will transfer through the SC this way.

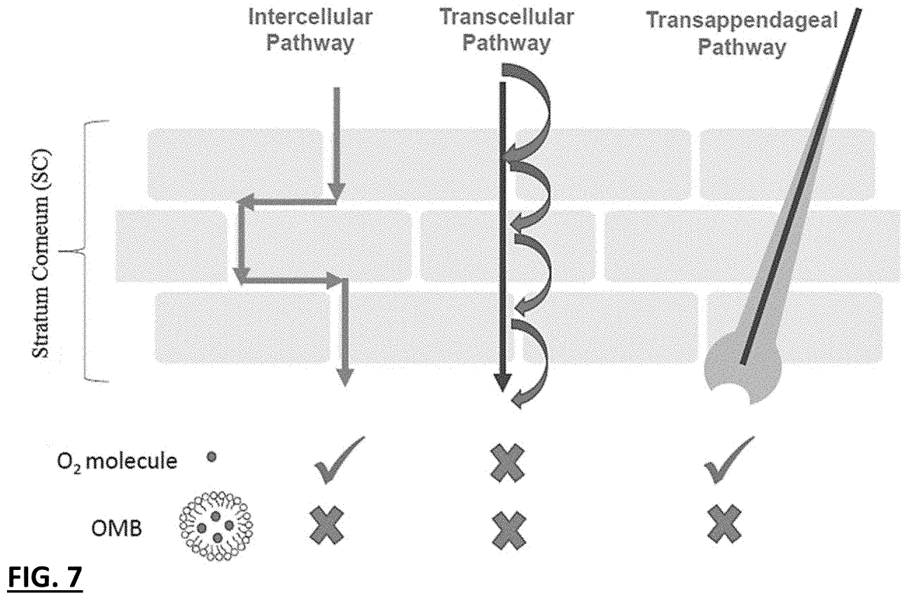

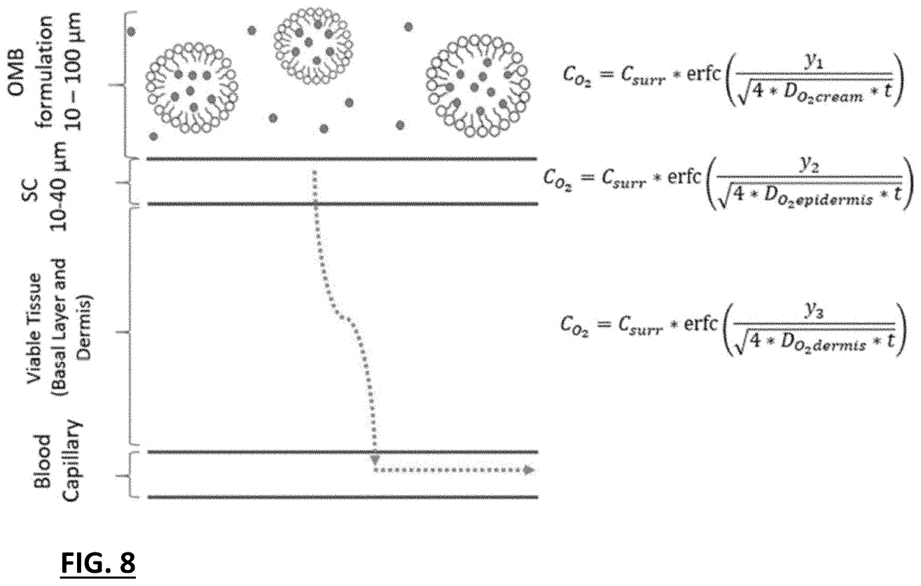

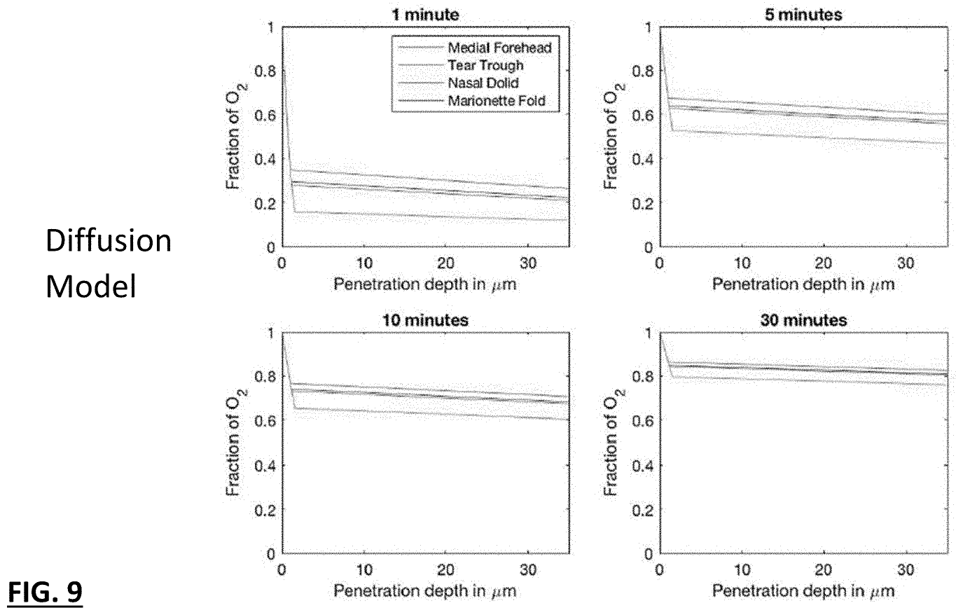

Although larger OMBs as a whole, with their lipid shell, are not expected to substantially diffuse through the SC to their target, oxygen is a small molecule that is expected to enter the skin intercellularly and through the transappendageal pathway. The diffusion of oxygen from the OMBs to the peritoneum, the muscle-tissue lining of the abdominal cavity, is well-documented and has been modeled theoretically and studied in vivo, justifying the use of OMBs to deliver oxygen directly to tissues. Literature also exists on oxygen diffusion through human skin which estimates mass transfer coefficients and partial pressures of the layers of skin. Thus, in various embodiments, it is proposed that the topical application of an OMB formulation and/or cosmeceutical can allow oxygen and/or other compounds to penetrate the stratum corneum via one or more pathways into the skin, including by (1) intercellular pathways, (2) transcellular pathways, and/or (3) transappendageal pathways (see FIG. 7). These pathways allowed Oxygen to penetrate and diffuse through the skin layers, as best shown in FIGS. 8 and 9.

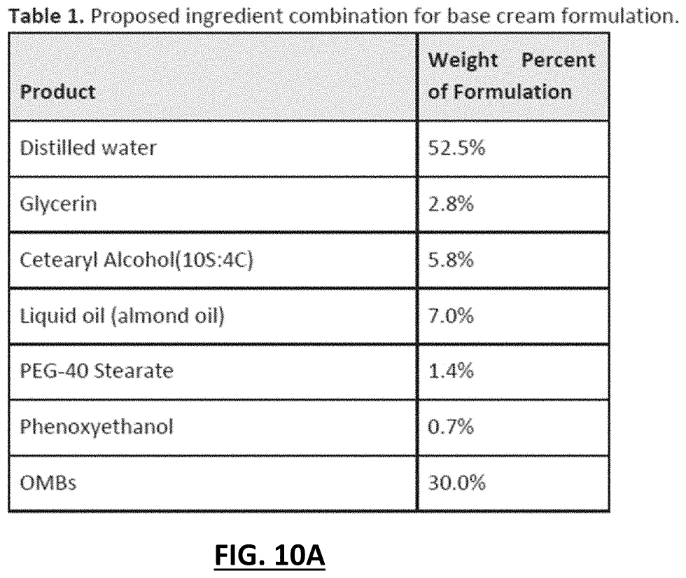

In various embodiments, some of all a variety of compounds may be incorporated into the OMB formulation, including solvents, emollients, humectants, emulsion stabilizers and/or preservatives. After analyzing a variety of common ingredients used in cosmetic skin care products, an initial exemplary ingredient formulation was proposed (see FIG. 10A). Each ingredient in this formulation desirably serves as an important contribution to the topical skin cream. Distilled water acts as the solvent, carrier, and diluent for the skin cream by helping dissolve the ingredients used in the formulation. Glycerin, a common ingredient in skin care products, acts as an emollient and humectant; it prevents water loss by forming a coat at the top of the skin to keep it soft and moisturized, and it draws and retains water into the top of the skin surface. Cetearyl alcohol comprises of stearyl alcohol and cetyl alcohol, which act as surfactants, reducing the surface tension of the mixture. In addition, they act as a viscosity increasing agents. Stearyl alcohol acts as a lubricant and cetyl alcohol acts as a thickening agent.

The weight percent composition of various ingredients in the alternative topical cream formulations can be in accordance with the disclosure of U.S. Pat. No. 5,153,230, entitled “Topical Skin cream Compositions” by Jeffrey H. Manzoor, the disclosure of which is incorporated by reference herein in its entirety. According to this patent, a formulation for prevention and treatment of aging skin can include ingredients in the ranges of 5-12 wt % of Stearyl alcohol, and about 2-5 wt % of cetyl alcohol. Glycerin can desirably be effective up to 18 wt %, with a wt % ranging from 2-12% being preferred. Phenoxyethanol, which can irritate the skin in higher concentrations, may be included in concentrations at or below 2.2%, and can be particularly effective at 1 wt %.

These proposed ingredients can desirably serve as a base cream formulation, with a variety of other compounds potentially added for a variety of reasons. For example, if a longer shelf life for the cream is desirous, a preservative such as phenoxyethanol may be added, which acts by preventing the growth of disease-causing organisms. In addition, the smell or texture of the cream may be important, which may mandate additional additives as required.

In various alternative embodiments, a base cream or other composition formulation containing OMBs can be manufactured and stored at lower than ambient temperatures, which may include refrigerated, freezing and/or subfreezing temperatures depending upon the formulation components, packaging and/or desired durability and/or “shelf-life.” In some embodiments, the formulation may be manufactured, stored, transported and/or distributed via a “cold chain” storage and deliver system during the entire production, storage and/or use cycle (i.e., a cold chain storage and delivery of an OMB formulation for skincare), or in other embodiments some of the components of the formulation may be stored and/or distributed at ambient or elevated temperatures prior to preparation and/or mixing of various components to create an OMB formulation, at which time the mixed formulation maybe maintained at ambient and/or cooled temperatures until use.

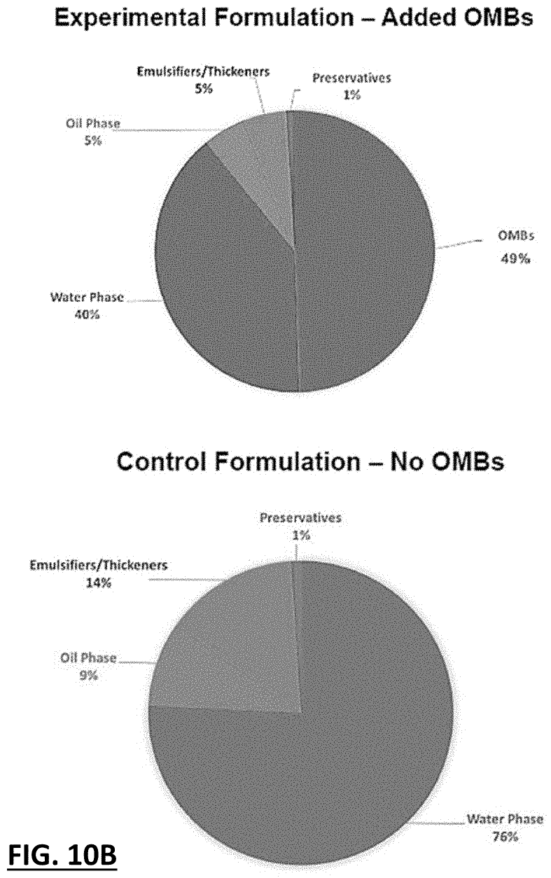

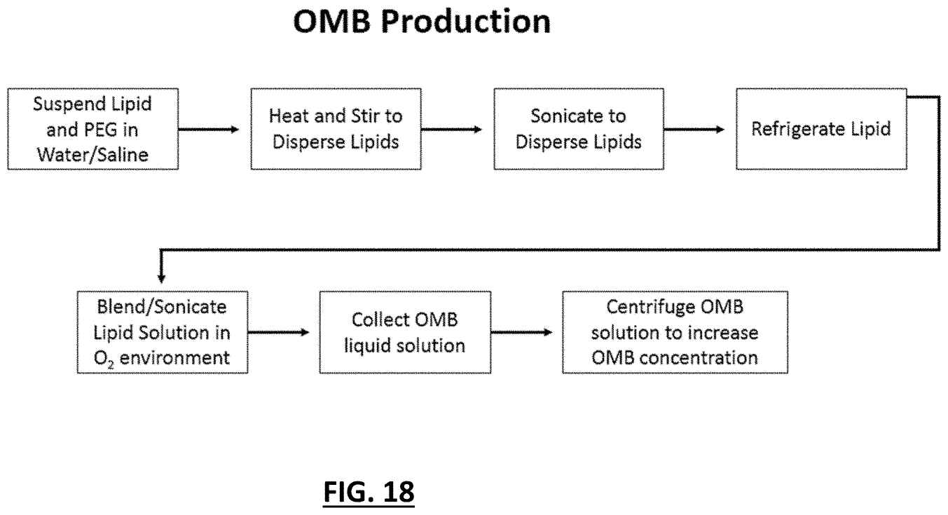

In one exemplary embodiment, the experimental topical formulation was created, with a test version containing OMBs and a control version having no OMBs therein (see FIG. 10B). A variety of formulation methods was utilized, including mixing and heating different groups of the ingredients separately (see FIG. 11), and then combining all of the groups together. Another exemplary embodiment of producing an OMB lotion is depicted in the flowchart of FIG. 19.

More specifically, one exemplary formulation included different groups consisting of related ingredients added together. First, part A, the water phase, was heated together into a beaker up to 75 degrees C. for 15 minutes. Next, part B, the oil phase, and part C, the emulsifiers/thickeners, were heated independent of each other to 75 degrees C. Once both part B and C were at 75 degrees C., they are mixed together, and part A is then slowly poured in while stick blending until a homogenous cream forms and thickens. After the cream forms, part D, phenoxyethanol, is added into the mixture and blended using a homogenizer, while cooling to a temperature of 40 degrees C. In this embodiment, this temperature is the preferred “highest” temperature in which the OMBs, part E, can be added while maintaining stability. The OMBs are added to the cream mixture at 40 degrees C. and are continuously blended until viscosity reaches a desired specification. In a preferred embodiment, a 30:70 OMB-to-cream ratio by volume is desired, although higher and lower ratios may be utilized with varying results. The preferred embodiment of 30:70 will give a total initial oxygen content of about 20%, shown clinically to be effective in skin anti-aging.

Depending on the outcome of the cream's density and viscosity, the composition of the cream formulation can be altered, with a “trial and error” approach potentially necessary to produce a cream with a satisfactory density and viscosity, depending upon other additives in the cosmeceutical. In addition, the oxygen will desirably be retained in the microbubbles within the formulation, but the destruction of the microbubbles may be possible during the mixing process, which can be another point of unanticipated variability depending upon the desired ingredients and/or proposed production processes and/or volumes (i.e., continuous and/or batch manufacturing).

One alternative OMB manufacturing method may include the use of a sonicator or other microbubble manufacturing device/technique utilized directly in the cosmeceutical cream in an oxygen or other environment (i.e., direct oxygen sonication into the cream), resulting in a cream infused with oxygen bubbles. This potential alternative production method for the microbubbles could include the use of lecithin to interact with the oxygen to trap it and form micelles in the cream.

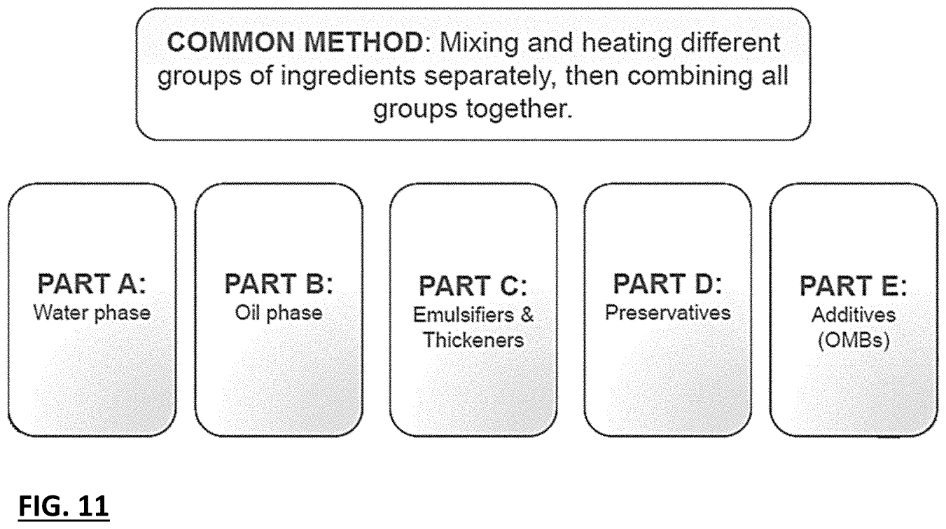

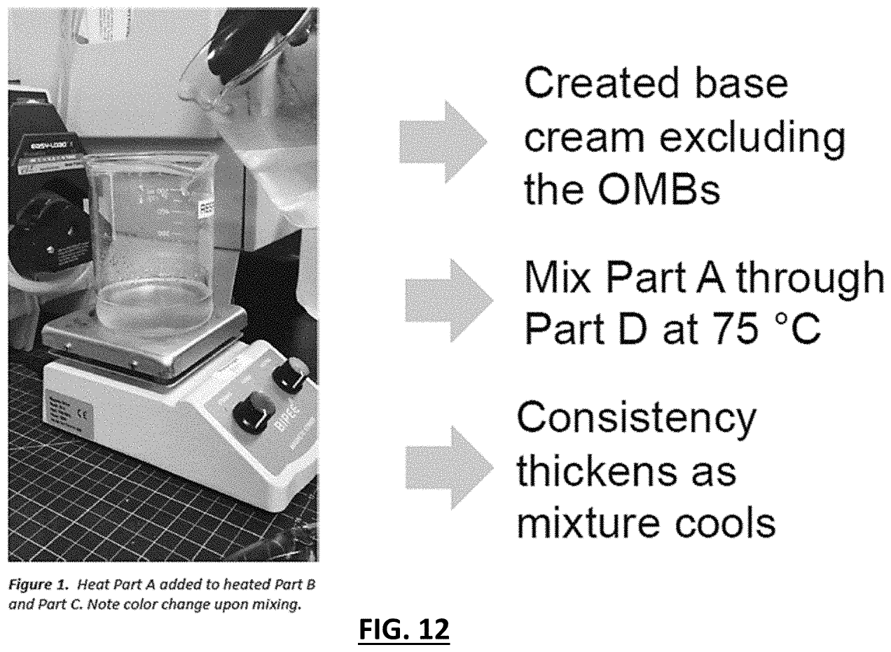

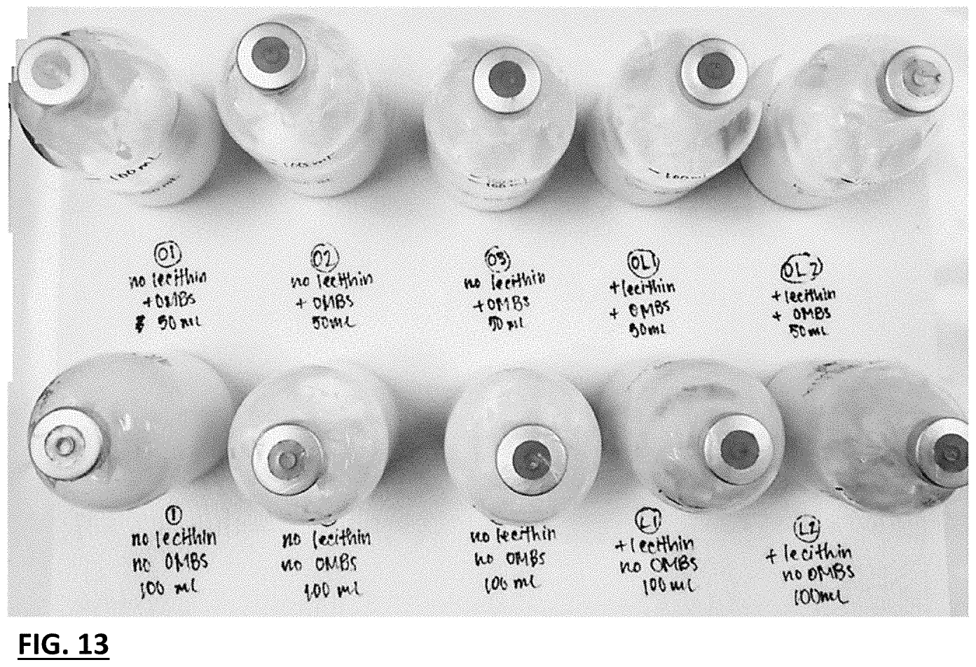

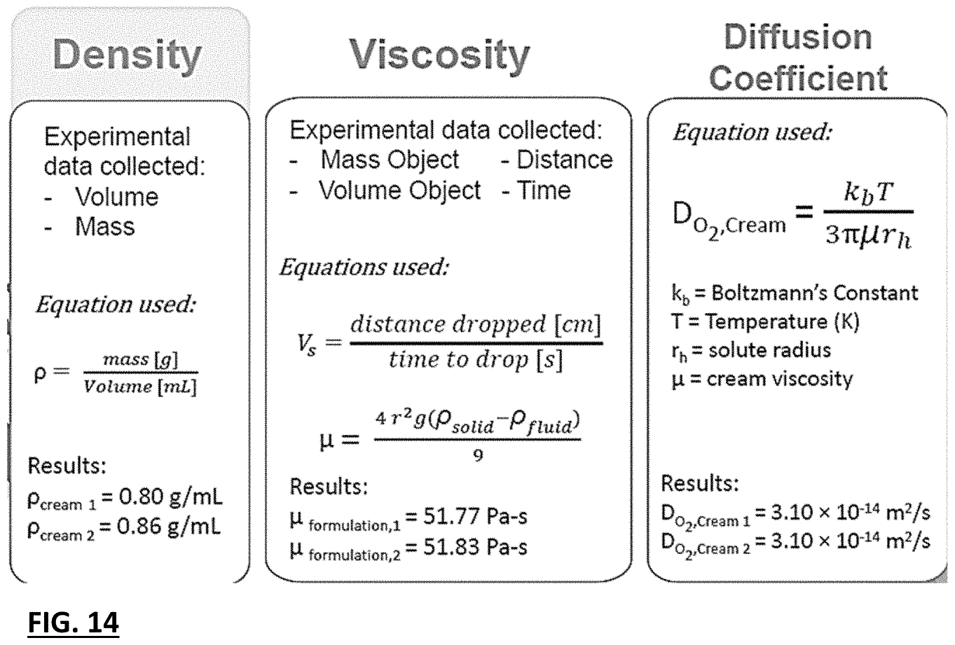

FIG. 12 depicts one exemplary embodiment of a topical cream, and FIG. 13 depicts five experimental samples (top row) to which OMBs were added and five control samples (bottom row) with no added OMBs, each vial totaling 100 ml. Each condition had three samples of the formulation with one emulsion stabilizer (left three bottles of each row), and two samples of the formulations with two emulsion stabilizers (right two bottles of each row). Experimental results of these formulations are provided in FIGS. 14 and 15, and most notably show a significant increase in Oxygen penetration depth as compared to the depths of FIG. 9.

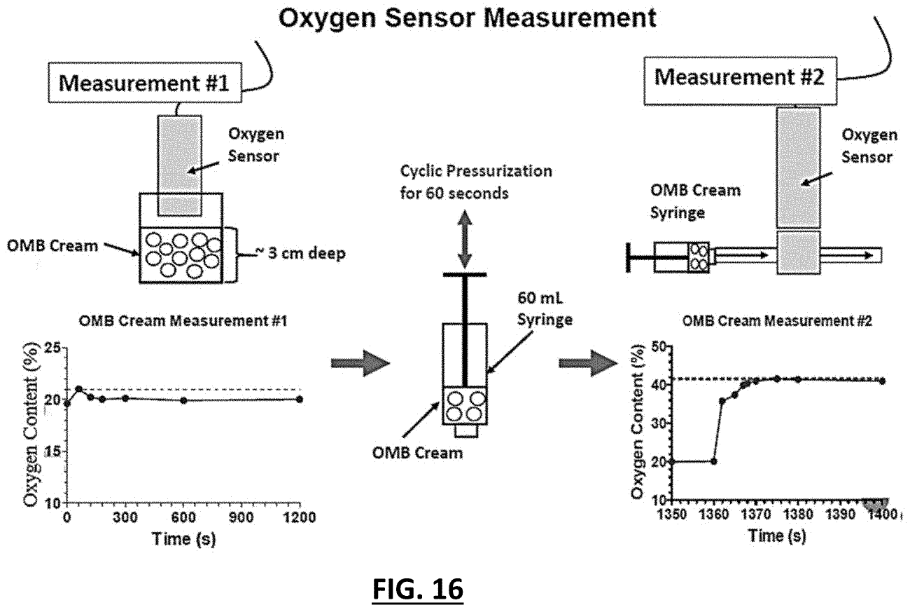

FIG. 17 depicts emulsion stability for the two OMB formulations, wherein formulation 1 comprised a stable cream at room temperature of 25 C, but degraded to a liquid at Refrigerator temperature of 4 C, while formula two remained a stable cream at all temperatures.

In one preferred embodiment, an OMB-infused cream or other cosmeceutical can be used every day by a consumer to prevent wrinkles and act as an anti-aging topical. The consumer will desirably apply the topical at an optimal thickness to achieve oxygen release, and in some embodiments may utilize a gas impermeable mask or other layering material over the applied OMB cream to enhance skin absorption of the oxygen. Such a physical barrier is intended to restrict oxygen diffusion into the air after application, aiding in higher oxygen content diffusion into the skin. Alternatively, relatively high levels of oxygen in alternative creams and/or additional sealant additives may be included in “higher performing” cream formulations. Based on the ingredients in the OMB-stabilizing cream, the cream could potentially be designed so that the underlying skin layer(s) react in a way that would promote oxygen transport into the epidermis rather than being released to the surrounding atmosphere. If desired, a non-toxic skinning or hardening agent could be incorporated into the cosmeceutical to isolate the material from the atmosphere while maintain the cosmeceutical in a liquid or cream form, in a manner similar to the inclusion of styrene or parrafin wax in polyester resins.

### Experimental Results

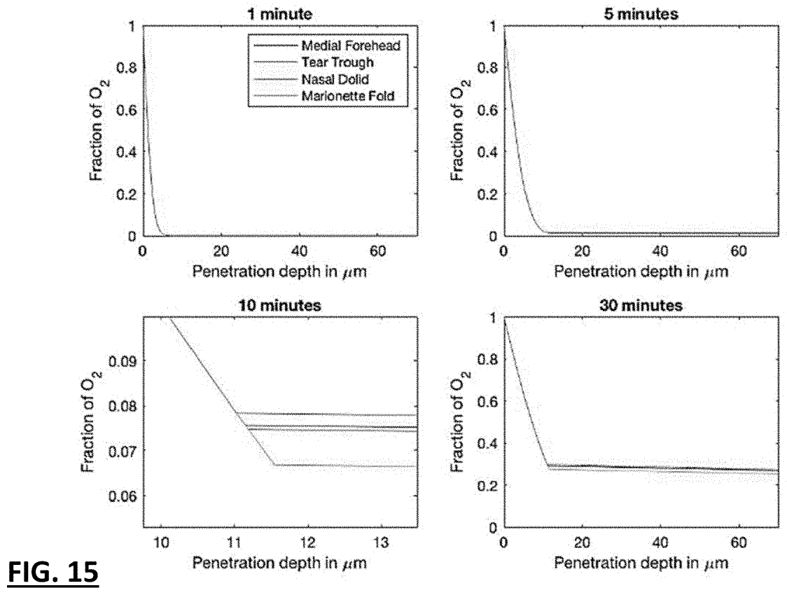

FIG. 16 depicts an exemplary microbubble stability test, wherein an OMB enriched topical cream was placed in a 50 mL beaker open to an air environment. An oxygen sensor was placed directly above the beaker so that passive oxygen being released from the cream can be detected. Immediately after OMB-cream placement in the beaker, a jump of 1% oxygen content was seen, with an oxygen content of ^{˜}21-20% being seen for the next 20 min (1200 seconds). The OMB cream was then extracted out of the beaker and into a 60 mL syringe. The syringe plunger was repeatedly moved inward and outward for 60 s to help break down the cream, with the cream appearing to remain structurally stable after this cyclic loading. The syringe was then connected to a flow-through fixture that attaches to the oxygen sensor. The resulting cyclically-loaded OMB-cream was pushed through the flow through fixture, with a jump of ^{˜}20% in oxygen content being observed within 5 seconds, with a total oxygen content of ^{˜}41% maintained for the remainder of the measurements.

This test demonstrated that the OMB-enriched topical skin care cream had exceptional resistance to oxygen diffusion when the volume-to-surface area ratio was high (V/A ^{˜}0.7 for Measurement #1 sample)—i.e., when the volume-to-surface area ratio is high, the characteristic oxygen diffusion length is large enough that it takes a significant amount of time to release the oxygen through the cream. In order to expedite the release of oxygen out of the OMB-cream emulsion, therefore, cyclic pressurization was used to mechanically disrupt the structural integrity of the OMB-cream foam, allowing for the timely release of oxygen out of the cream. The testing results confirmed that storing the cream in an oxygen-enriched large container (high V/A) will cause the OMB-cream to remain relatively stable. Upon application of the cream across the skin in a relatively thin layer, the volume-to-surface area ratio is drastically decreased leading to a great reduction in the characteristic oxygen diffusion length which allows for the expedited release of oxygen to the skin. Thus, the OMB cream can be stored at high V/A and then utilized at low V/A.

If desired, the OMB's utilized herein could include a liquified slurry of OMBs created by generating a OMB solution with an approximately 60% void fraction (60% oxygen microbubbles, 40% liquid) within a carrier solution such as saline.

DRUG Delivery

In various embodiments, the topical application of OMBs and/or other microbubble formulations may enhance and/or facilitate the delivery and/or absorption of oxygen (or reverse transfer of carbon dioxide) and/or may enhance and/or facilitate the delivery of other compounds and/or medications in local and/or systemic manners. For example, OMBs and/or other microbubble formulations may be particularly useful in delivering cannabinoids and/or similar substances to an individual, including the psychoactive Δ⁹-tetrahydrocannabinol (THC) and the non-psychoactive cannabidiol (CBD), commercially available as pharmaceutical formulations such as Nabiximols (Sativex®—a commercially available oromucosal spray that contains a mixture of THC and CBD) and Dronabinol (Marinol®), an oral preparation of synthetic THC. In addition, the phospholipid monolayer variation of microbubbles described herein may have particular affinity and usefulness in conjunction with the lipid-soluble cannabinoids THC and CBD, as the topical co-administration of lipids may increase absorption and/or bioavailability of THC in mammals by more than 2.5-fold, and of CBD by almost 3-fold (which profound increase in systemic exposure may significantly affect the therapeutic effects or toxicity of these cannabinoids).

In various embodiments, a microbubble formulation may serve as a carrier to transfer THC and CBD to the systemic circulation via the lymphatic system following topical application with lipids. Drugs that are transported via the lymphatic system can avoid hepatic first-pass metabolism and therefore achieve significantly higher bioavailability than after administration in lipid-free formulation. Thus, co-administration of microbubble lipids may substantially increase the systemic exposure to cannabis or cannabis-based medicines, and testing suggests that one primary mechanism of the increased absorption of cannabinoids in the presence of lipids may be lymphatic transport. Desirably, an amount of lipid present in the microbubble formulation could be sufficient to “humidify” and/or soften the skin surface and promote the absorption of cannabinoids, thereby increasing the potential systemic exposure to cannabinoids. The increase in systemic exposure to cannabinoids in humans is of potentially high clinical importance as it could turn a barely effective dose of topically administered cannabis into a highly effective one, or be a mechanism for adjustment of effective therapeutic dose.

OMB Formulation Delivery & Packaging

In various embodiments, the OMB formulations describe herein can be manufactured, stored and/or delivered in a variety of manners and packaging, including in resealable and/or disposal, single-use packaging. In at least one exemplary embodiment, an OMB formulation can be manufactured and packaged in airtight packaging, with the formulation capable or remaining in a stable and usable condition for an extended period of time, such as up to 2 years or longer. Desirably, the packaging will allow the OMB formulation to remain fully sealed until the time of application, when the seal can be broken and the formulation applied quickly thereafter.

If desired, an OMB storage and delivery device could include multiple reservoirs for containing materials, including OMB formulations, which may allow for sequential application and/or allow for pre-mixing of contents prior to application. For example, it may be desirous to humidify and/or “wet” the skin surface prior to topical application to desirably facilitate the durability of the OMBs and/or the absorption of oxygen into the skin. In such case, the OMB storage and delivery device could include a first reservoir containing a moisturizing agent containing a lipid or gel (or other commonly accepted moisturizing agent), and a second reservoir containing the OMB formulation, with the individual first applying the moisturizer and then subsequently applying the OMB formulation. In another embodiment, the reservoirs might be combinable prior to application. This arrangement could allow the OMB formulation to remain relatively stable for transport, with mixing occurring immediately prior to use.

In various embodiments, the application of an OMB formulation could include situations where the OMB formulation is completely absorbed by the skin and/or naturally “sloughed” off with discarded skin cells, while in other embodiment may be “temporarily” applied to the surface of the skin and then intentionally removed (via natural and/or artificial techniques such as washing) from the skin surface after a period of time. In another exemplary embodiment the OMB formulation might comprise a wash or splashing agent, or even an aerosolized agent in some embodiments.

Microbubble Production

Oxygen microbubbles can be formulated with either a lipid monolayer shell, a biocompatible polymer shell, or a combination thereof. In addition to oxygen, the shell-stabilized microbubbles can be prepared with a variety of therapeutic gases. Additionally, these microbubbles can be formulated in a variety of biocompatible fluids that act as the continuous phase liquid for microbubble suspension. The lipids which may be used to prepare the gas and gaseous precursor filled microspheres used in the present invention include but are not limited to: lipids such as fatty acids, lysolipids, phosphatidylcholine with both saturated and unsaturated lipids including dioleoylphosphatidylcholine; dimyristoylphosphatidylcholine; dipentadecanoylphosphatidylcholine; dilauroylphosphatidylcholine; dipalmitoylphosphatidylcholine (DPPC); distearoylphosphatidylcholine (DSPC); phosphatidylethanolamines such as dioleoylphosphatidylethanolamine and dipalmitoylphosphatidylethanolamine (DPPE); phosphatidylserine; phosphatidylglycerol; phosphatidylinositol; sphingolipids such as sphingomyelin; glycolipids such as ganglioside GMI and GM2; glucolipids; sulfatides; glycosphingolipids; phosphatidic acids such as dipalymitoylphosphatidic acid (DPPA); pabnitic acid; stearic acid; arachidonic acid; oleic acid; lipids bearing polymers such as polyethyleneglycol, i.e., PEGylated lipids, chitin, hyaluronic acid or polyvinylpyrolidone; lipids bearing sulfonated mono-, di-, oligo- or polysaccharides; cholesterol, cholesterol sulfate and cholesterol hemisuccinate; tocopherol hemisuccinate; lipids with ether and ester-linked fatty acids; polymerized lipids (a wide variety of which are well known in the art); diacetyl phosphate; dicetyl phosphate; stearylamine; cardiolipin; phospholipids with short chain fatty acids of 6-8 carbons in length; synthetic phospholipids with asymmetric acyl chains (e.g., with one acyl chain of 6 carbons and another acyl chain of 12 carbons); ceramides; non-ionic liposomes including niosomes such as polyoxyethylene fatty acid esters, polyoxyethylene fatty alcohols, polyoxyethylene fatty alcohol ethers, polyoxyethylated sorbitan fatty acid esters, glycerol polyethylene glycol oxystearate, glycerol polyethylene glycol ricinoleate, ethoxylated soybean sterols, ethoxylated castor oil, polyoxyethylene-polyoxypropylene polymers, and polyoxyethylene fatty acid stearates; sterol aliphatic acid esters including cholesterol sulfate, cholesterol butyrate, cholesterol iso-butyrate, cholesterol palmitate, cholesterol stearate, lanosterol acetate, ergosterol palmitate, and phytosterol n-butyrate; sterol esters of sugar acids including cholesterol glucuroneide, lanosterol glucuronide, 7-dehydrocholesterol glucuronide, ergosterol glucuronide, cholesterol gluconate, lanosterol gluconate, and ergosterol gluconate; esters of sugar acids and alcohols including lauryl glucuronide, stearoyl glucuronide, myristoyl glucuronide, lauryl gluconate, myristoyl gluconate, and stearoyl glucon-ate; esters of sugars and aliphatic acids including sucrose laurate, fructose laurate, sucrose palmitate, sucrose stearate, glucuronic acid, gluconic acid, accharic acid, and polyuronic acid; saponins including sarsasapogenin, smilagenin, hederagenin, oleanolic acid, and digitoxigenin; glycerol dilaurate, glycerol trilaurate, glycerol dipalmitate, glycerol and glycerol esters including glycerol tripalmitate, glycerol distearate, glycerol tristearate, glycerol dimyristate, glycerol trimyristate; longchain alcohols including n-decyl alcohol, lauryl alcohol. myristyl alcohol, cetyl alcohol, and n-octadecyl alcohol; 6-(5-cholesten-3 yloxy)-1-thio-D-galactopyranoside; digalactosyldiglyceride; 6-(5-cholesten-3-yl oxy)hexyl-6-amino-6-deoxy-1-thio-D-galactopyranoside; 6-(5-cholesten-3-yloxy)hexyl-6-amino-6-deoxyl-1-thio-a-D-mannopyranoside; 12-(((7′-diethylarninocoumarin-3-yl)carbonyl)methylamino)-octadecanoic acid; N-[12-(((7′-diethylaminocoumarin-3-yl) carbonyl)methyl-amino) octadecanoyl]-2-aminopalmiticacid; cholesteryl) 4T-trimethylammonio)butanoate; N-succinyldioleoylphosphatidylethanolamine; 1,2-dioleoyl-sn-glycerol; I,2-dipalmitoyl-sn-3-succinylglycerol; 1,3-dipalmitoyl-2-succinylglycerol; I-hexadecyl-2-palmitoyl glycerophosphoethanolamine and palmitoylhomocysteine, and/or combinations thereof.

If desired, a variety of cationic lipids such as DOTMA, N-[1-(2,3-dioleoyloxy)propyl]-N,N,N-trimethylammoium chloride; DITTAP, 1,2-dioleoyloxy-3-(trimethylammonio) propane; and DOTB, 1,2-dioleoyl-3-(4′-trimethyl-ammonio) butanoyl-sn-glycerol may be used. In general the molar ratio of cationic lipid to non-cationic lipid in the liposome may be, for example, 1:1000, 1:100, preferably, between 2:1 to 1:10, more preferably in the range between 1:1 to 1:2.5 and most preferably 1:1 (ratio of mole amount cationic lipid to mole amount non-cationic lipid, e.g., DPPC). A wide variety of lipids may comprise the non-cationic lipid when cationic lipid is used to construct the microsphere. Preferably, this non-cationic lipid is dipalmitoylphosphatidylcholine, dipalmitoylphosphatidylethanolamine or dioleoylphosphati-dylethanolamine. In lieu of cationic lipids as described above, lipids bearing cationic polymers such as polylysine or polyarginine, as well as alkyl phosphonates, alkyl phosphinates, and alkyl phosphites, may also be used to construct the microspheres.

In at least one exemplary embodiment, more preferred lipids can be phospholipids, preferably DPPC, DPPE, DPPA and DSPC, and most preferably DSPC.

In addition, examples of saturated and unsaturated fatty acids that may be used to prepare the stabilized micro-spheres used in the present invention, in the form of gas and gaseous precursor filled mixed micelles, may include molecules that may contain preferably between 12 carbon atoms and 22 carbon atoms in either linear or branched form. Hydrocarbon groups consisting of isoprenoid units and/or prenyl groups can be used as well. Examples of saturated fatty acids that are suitable include, but are not limited to, auric, myristic, palmitic, and stearic acids; examples of unsaturated fatty acids that may be used are, but are not limited to, lauroleic, physeteric, myristoleic, palmitoleic, petroselinic, and oleic acids; examples of branched fatty acids that may be used are, but are not limited to, isolauric, isomyristic, isopalmitic, and isostearic acids. In addition, to the saturated and unsaturated groups, gas and gaseous precursor filled mixed micelles can also be composed of 5 carbon isoprenoid and prenyl groups.

The biocompatible polymers useful as stabilizing compounds for preparing the gas and gaseous precursor filled microspheres used in the present invention can be of either natural, semi-synthetic or synthetic origin. As used herein, the term polymer denotes a compound comprised of two or more repeating monomeric units, and preferably 10 or more repeating monomeric units. The term semi-synthetic polymer, as employed herein, denotes a natural polymer that has been chemically modified in some fashion. Exemplary natural polymers suitable for use in the present invention include naturally occurring polysaccharides. Such polysac charides include, for example, arabinans, fructans, fucans, galactans, galacturonans, glucans, mannans, xylans (such as, for example, inulin), levan, fucoidan, carrageenan, galatocarolose, pectic acid, pectin, amylose, pullulan, glycogen, amylopectin, cellulose, dextran, pustulan, chitin, agarose, keratan, chondroitan, dermatan, hyaluronic acid, alginic acid, xanthan gum, starch and various other natural homopolymer or heteropolymers such as those containing one or more of the following aldoses, ketoses, acids or amines: erythrose, threose, ribose, arabinose, xylose, lyxose, allose, altrose, glucose, mallllose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof. Exemplary semi-synthetic polymers include carboxymethylcellulose, hydroxymethylcellulose, hydroxypropylmethylcellulose, methylcellulose, and methoxycellulose. Exemplary synthetic polymers suitable for use in the present invention include polyethylenes (such as, for example, polyethylene glycol, polyoxyethylene, and polyethylene terephthlate), polypropylenes (such as, for example, polypropylene glycol), polyurethanes (such as, for example, polyvinylalcohol (PVA), polyvinylchloride and polyvinylpyrrolidone), polyamides including nylon, polystyrene, polylactic acids, fluorinated hydrocarbons, fluorinated carbons (such as, for example, polytetrafluoroethylene), and polymethylmethacrylate, and derivatives thereof. Methods for the preparation of such polymer-based microspheres will be readily apparent to those skilled in the art, once armed with the present disclosure, when the present disclosure is coupled with information known in the art, such as that described and referred to in Unger, U.S. Pat. No. 5,205,290, the disclosures of which are hereby incorporated herein by reference, in their entirety.

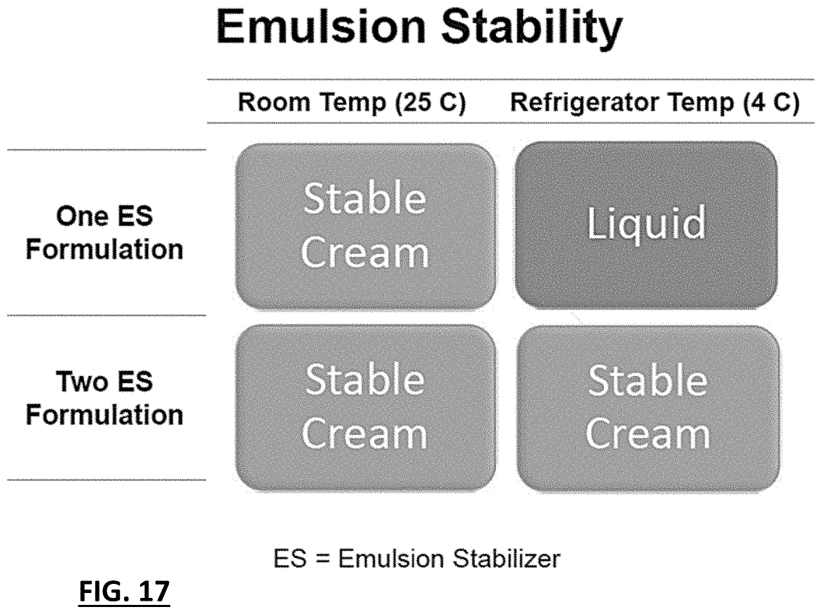

One exemplary method of producing OMB's is depicted in the flow chart of FIG. 18. In another exemplary embodiment, oxygen microbubbles can be produced by mixing lipids at a 9:1 molar ratio of distearoyl phosphatidylcholine (DSPC) to poly(ethylene glycol)-40 stearate (PEG40S) in saline and sonicated at low power to create the small, unilamellar liposomes. O2 and liposomes (5 mg/mL) are then combined in the reaction chamber, where a high-power, ½-inch diameter, 20-kHz sonicator tip emulsifies the oxygen gas into micrometer-scale spheres around which phospholipid adsorbs from vesicles and micelles and self-assembles into a highly condensed (solid) monolayer coating. OMBs can be separated from macroscopic foam in a subsequent flotation container and collected in syringes and centrifuged (500 g for 3 min) to form concentrated OMBs. The sonication chamber and container are jacketed with circulating coolant to maintain a constant temperature of 20° C.

A desired OMB size distribution can be varied by choosing different residence times in the flotation container (e.g., 153 min for a 10-μm diameter cut-off; 38 min for a 20-μm diameter cut-off). Size distribution can be measured, for example, by electrical capacitance, light extinction/scattering, flow cytometry scatter, and optical microscopy. Alternatively, size selection may be unnecessary and may be removed from the process. OMB volume fraction is measured, for example, by gravimetric analysis and varied from 20-90 vol % by dilution with saline. Microbubble size and concentration is measured over time to investigate coalescence, Ostwald ripening and stability in storage.

Skin Recovery Foams

In some embodiments, a material such as a high gas particle concentration monolayer-stabilized microfoam (i.e., OMB foam or emulsion) can be incorporated into a cream or lotion that can improve gas, drug, molecule and/or particle (or various combinations thereof) penetration, transfer and/or removal within and/or around the epidermis, the dermis and/or other anatomical regions proximate to a skin surface. More specifically, a desirable monolayer-stabilized microfoam can comprise monolayer-stabilized oxygen gas-in-liquid particles that exist at diameters ranging from 0.5 to 100 um in diameter. These micron-sized gas-in-liquid particles can be concentrated to a specific concentration or concentration range thereby creating a microfoam/microemulsion containing a predetermined or desired gas-to-liquid ratio. These microparticles can be concentrated at virtually any volume (0.1 to >1,000,000 mL) to produce a microfoam with a gas-to-liquid ratio ranging anywhere from 1:20 up to 99:1. The resulting bulk microfoam material can then be delivered within or around the epidermis and dermis to desirably act as a gas phase couplant which improves gas transfer to, within and from the skin and/or underlying anatomy. The gases described herein that can be used individually or as a combination could be, but not limited to, oxygen, ozone, nitrogen, butane, propane, fluorocarbons, etc. Additionally, pharmaceuticals and therapeutic agents can be attached and/or incorporated into the monolayer-stabilized gas microfoam (or can comprise liquid, gas or solid additives to saline or other carriers thereof) to further improve epidermal and dermal recovery and appearance.

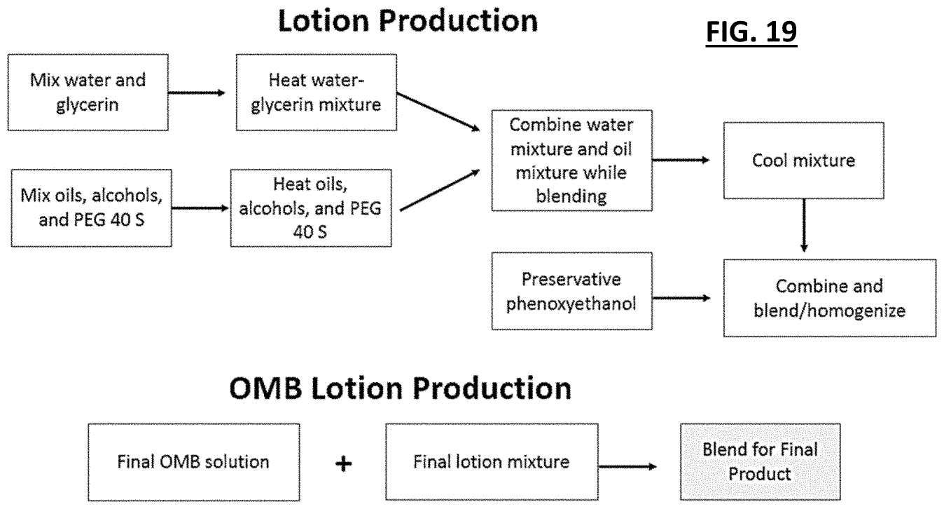

In various embodiment, a monolayer-stabilized gas-in-liquid microfoam can be prepared to desirably enhance the transport of and/or remove of a predetermined gas, drug, molecule, particle, or combination species from an anatomical target area such as a skin surface. In many embodiments, the technology can utilize a topical application in combination with gas and particle diffusion between the topical substance and the skin surface to transport and/or remove unwanted species from the epidermis and dermis to enhance recovery and health of the skin. Specifically, a gas microfoam can be prepared so that it contains 50-100% of a single or combination gas mixture by gas volume so that diffusion drives that species into the tissue (see FIG. 20). Desirably, diffusion forces can “drive” a targeted gas species (or other components) from a region of higher gas concentration 2010 to a region of lower gas concentration 2020 (i.e., from the topical microfoam into the epidermis and/or potentially the dermis, until there is no longer a sufficient concentration gradient from the microfoam to the anatomy to cause diffusion through the intervening anatomical layers. The microfoam, when administered to a patient, can be mathematically modelled as a “non-enclosed” gas, particle, and/or pharmaceutical source, because the material desirably does not require an enclosed chamber to deliver the gas and other possible actives into the skin surface. In some instances, upon application the microfoam lotion may form a desiccated surface “skin” layer or “vapor barrier” which can greatly inhibit release or diffusion of contained gas to the surrounding atmosphere, while maintaining a steady diffusive flow into the skin layers.

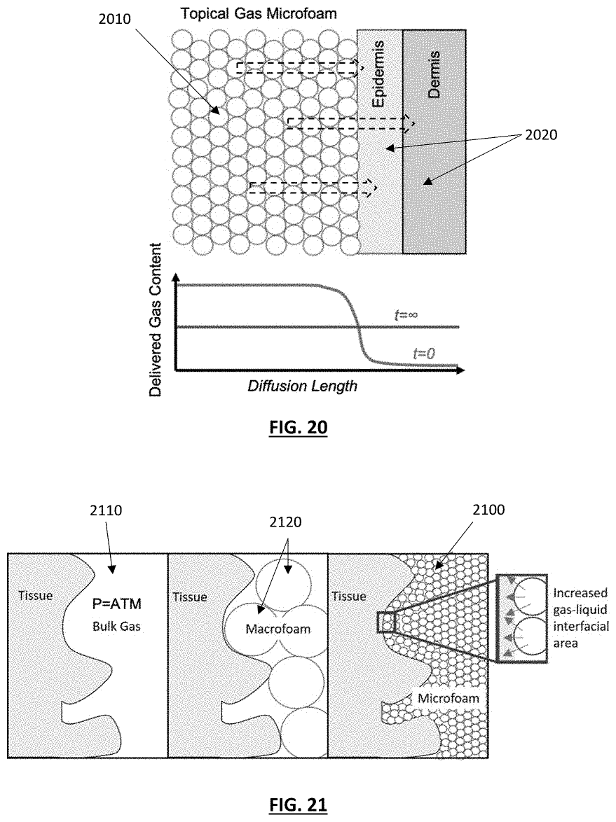

In one exemplary embodiment, the gas-in-liquid microfoam structure can comprise a plurality of monolayer stabilized gas microbubble particles that are packed together to form a stable microfoam structure. These micron-sized particles that can make up the microfoam can grant the composition unusual benefits to the underlying skin that are not conferred by macrofoam formulations or procedures that seek to introduce large amounts of non-enclosed, non-pressurized topical gases (i.e., hyperbaric oxygen therapy). As best shown in FIG. 21, gas microparticles 2100 that comprise the microfoam can wet and spread across a skin surface interface in a much more effective and densely packed manner much more effectively due to their small size and diameter. The micron-sized gas-in-liquid foam bubbles provide a dramatically heightened gas-liquid interfacial area (i.e., more skin surface area in direct contact with bubble surface area) between the skin and the microbubbles surface that increases the rate of gas transport between these two surfaces as compared to a relatively “flat” interface between a bulk gas 2110 and the skin, or the surface between relatively larger macrobubbles 2120 and the skin.

In the field of fermentation, gas microparticles (commonly referred to as microbubbles or colloidal gas aphrons) can be used as an additive in bioreactors to increase the rate of volumetric gas transport when increasing the rate of impeller mixing (i.e., shear) may not be desirable. Specifically, air gas microbubbles experimentally sparged in a test system led to a 6-fold increase in measured gas transfer for the fermentation of carbon monoxide by B. methylotrophicum. Similarly, microbubble sparging in the commercial production of Xanthan Gum can dramatically improve the mass transfer of oxygen and thus enabled a more energy-efficient production. While microbubbles may introduce more interfaces and resistance barriers for gas transport on a per-microbubble basis, the sheer number and ability of microbubbles to increase the gas-liquid interfacial area greatly overrides any added per-microbubble resistance and enhances overall gas transport.

Gas-in-liquid microparticles can greatly enhance the transport of a gas in a bioreactor for the enhancement of fermentation, so a similar advantage can be realized for the disclosed transfer of substances, such as oxygen and/or lids, into and/or out of a skin layer. The novel use of a monolayer-stabilized gas-in-liquid microparticle for enhanced gas transport to and/or removal from the skin epidermis and dermis can greatly improve cosmetic procedure patient recovery. As one non-limiting example, the disclosed technology could be used to deliver oxygen to a patient who has just received a non-invasive, chemical, abrasive, or invasive skincare treatment by delivering a gas microfoam that comprises 95-100% oxygen gas with a lipid monolayer stabilizing shell to the patient's skin treatment area.

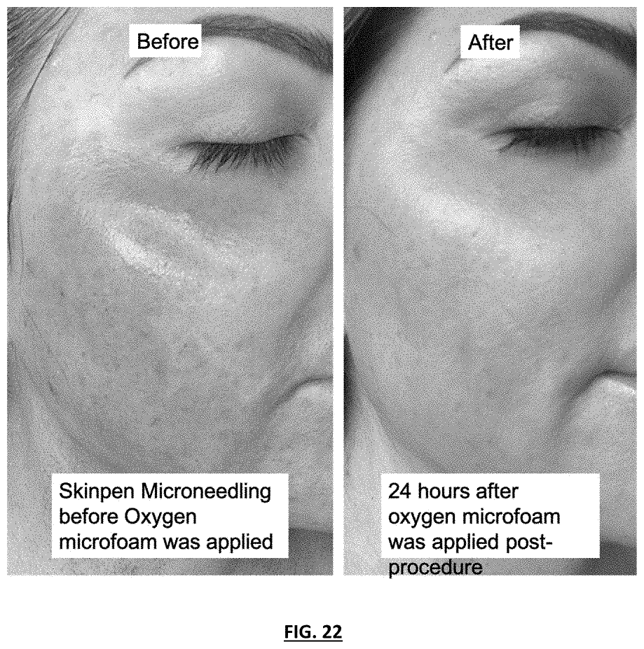

FIG. 22 depicts one experimental use of the disclosed microfoam creams containing oxygen and lipids, wherein a SkinPen® microneedling device (commercially available from Crown Aesthetics of 5005 Lyndon B Johnson Fwy Ste 370, Dallas, Tex. 75244, USA) was utilized to open channels to the dermis of a patient. The procedure causes irritation and significant reddening of the skin that typically stays highly visible on the patient's skin for a minimum of 3 days (i.e., 72 hours) or more (i.e., “Before” image at left of FIG. 22). However, after application of the closed oxygen gas microfoam lotion formulation to the area of treatment, a significantly accelerated healing reaction and loss of redness and irritation was observed after only 24 hours (i.e., “After” image at right of FIG. 22). In addition to improving the extent and rate of recovery, the accelerated healing disclosed herein can further lead to significant improvements in patient satisfaction including improvements in well-being, self-esteem, attractiveness measures, life satisfaction metrics, quality of life assessments and goal attainment as well as reducing patient anxiety and/or social phobias relating to the procedure or the underlying anatomical conditions themselves.

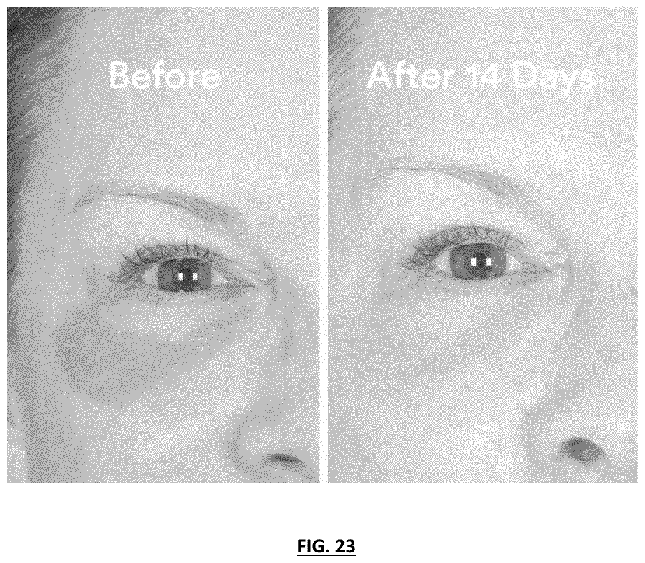

FIG. 23 depicts another experimental use of disclosed microfoam creams containing oxygen and lipids, wherein, a facial plastic surgeon performed a chemical peel on a patient's under-eye area, to desirably reduce some of the visible effects of aging. After the peel, the skin was very irritated and red (i.e., “Before” image at left of FIG. 23). The surgeon applied an oxygen gas microfoam moisturizing compound to the patient's treatment area. An amount of the oxygen gas microfoam moisturizing compound was also provided to the patient for self-administration, with the directions for the patient to apply the compound to the under-eye region for two times a day for two weeks. The patient returned for follow-up at the two-week point, at which appointment the patient presented an accelerated tissue recovery level (at two weeks) which was normally expected to occur at a minimum of 3 to 4 weeks post treatment (i.e., “After” image at right of FIG. 23).

These are a wide variety of compositions and/or compounds that may benefit from the incorporation of gas-lipid microfoams for the improved recovery and appearance of the epidermis and dermis after abrasive procedures to the skin. The gas phase of the microfoam can comprise a variety of single or combination gas mixture constituents that are delivered to the skin surface to promote healing. In addition, the gas phase of an exemplary microfoam may be designed with lower concentrations (up to and including 0% of a gas) so that the microfoam can desirably help diffuse unwanted gases within the epidermis and dermis out of the skin (such as, but not limited to, CO2 as one example).

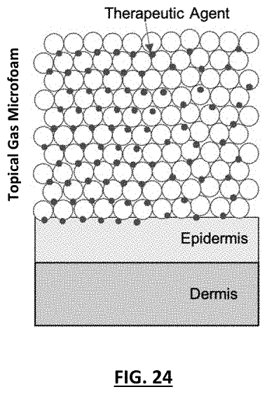

In addition to a therapeutic gas, the lipid monolayer stabilized gas microfoams described herein can be loaded or combined with pharmaceuticals or other therapeutic agents and/or particles. Specifically, a therapeutic payload could be incorporated into an aqueous phase, a gas phase and/or could be contained or attached to (or even form part of the shell of) the lipid monolayer of the microfoam (see FIG. 24). In one non-limiting embodiment, a therapeutic agent that could be incorporated into the microfoam could be hyaluronic acid, which desirably helps pull moisture towards the epidermis and dermis. The therapeutic agent could desirably be sized for transport through the epidermis, depending upon the objectives of the treatment and/or the specific of other procedures being performed. More invasive procedures which disrupt the surface skin layer might provide transport for larger molecules, whereas less and/or non-invasive procedures will typically allow the transport of smaller molecule therapeutics (<500 Daltons).

The present disclosure also expressly incorporates by reference herein the disclosure of U.S. Pat. No. 8,481,077 entitled “Microbubbles and Methods for Oxygen Delivery” to Kheir et al, filed Feb. 22, 2012; U.S. Pat. No. 10,058,837 entitled “Systems, methods, and devices for production of gas-filled microbubbles” to Borden et al, filed Aug. 26, 2010; and U.S. Pat. No. 10,124,126 entitled “Systems and methods for ventilation through a body cavity” to Borden et al, filed Apr. 18, 2014. The entire disclosure of each of the publications, patent documents, and other references referred to herein is incorporated herein by reference in its entirety for all purposes to the same extent as if each individual source were individually denoted as being incorporated by reference.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus intended to include all changes that come within the meaning and range of equivalency of the descriptions provided herein.

General

Many of the aspects and advantages of the present invention may be more clearly understood and appreciated by reference to the accompanying drawings. The accompanying drawings are incorporated herein and form a part of the specification, illustrating embodiments of the present invention and together with the description, disclose the principles of the invention.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the disclosure herein.

The various headings and titles used herein are for the convenience of the reader, and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

The use of the terms “a” and “an” and “the” and similar referents in the context of describing the invention are to be construed to cover both the singular and the plural, unless otherwise indicated herein or clearly contradicted by context. The terms “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

1. A method of accelerating the healing process of one or more damaged skin layers of an individual after a skin procedure, comprising contacting a surface of the one or more damaged skin layers of the individual with an aqueous formulation comprising microbubbles containing oxygen.

2. The method of claim 1, wherein the microbubbles are formulated from a lipid.

3. The method of claim 2, wherein the microbubbles are formulated from a lipid consisting of phosphatidylcholine.

4. The method of claim 2 wherein the lipid has a carbon chain length between 12 carbon atoms and 22 carbon atoms.

5. The method of claim 1, wherein the microbubbles are formulated from a polymer.

6. The method of claim 1, further comprising the step of applying the aqueous formulation to the surface skin layer.

7. The method of claim 1, further comprising the step of maintaining the aqueous formulation at a reduced temperature prior to delivery to the surface skin layer.

8. The method of claim 6, wherein the aqueous formulation is self-administered by the individual.

9. The method of claim 6, wherein a majority of the microbubbles in the aqueous formulation comprise substantially spherical gas-filled particles at or between 1 um to 1000 um in diameter in solution that are stabilized by an organic coating at the gas-liquid interface.

10. The method of claim 9, wherein the microbubbles are manufactured prior to application and stored for at least one day prior to application.

11. The method of claim 9, wherein the microbubbles are manufactured immediately prior to application.

12. The method of claim 6, further comprising applying an additional compound that modifies at least a portion of the one or more damaged skin layers of the individual prior to applying the aqueous formulation.

13. The method of claim 6, further comprising applying an additional compound that modifies at least a portion of the one or more damaged skin layers of the individual simultaneous to applying the aqueous formulation.

14. The method of claim 6, wherein the aqueous formulation further comprises an additional compound that modifies a surface dryness of at least a portion of the one or more damaged skin layers of the individual.

15. The method of claim 6, wherein the aqueous formulation further comprises at least one cannabinoid.

16. A method of delivering a gas to a subcutaneous region of an individual, comprising contacting a portion of one or more damaged skin layers of the individual with an aqueous formulation comprising microbubbles containing a gas.

17. The method of claim 16, wherein the microbubbles are formulated from a lipid.

18. The method of claim 16, wherein the microbubbles are formulated from a lipid monolayer.

19. The method of claim 16, wherein the microbubbles are formulated from a polymer.

20. A method of oxygenating one or more damaged skin layers of an individual, comprising contacting a surface of the one or more damaged skin layers with an aqueous formulation comprising microbubbles containing oxygen.

21. The method of claim 20, wherein the microbubbles are formulated from a lipid.

22. The method of claim 20, wherein the microbubbles are formulated from a polymer.
